(19) United States
(12) Patent Application Publication
(10) Pub. No.: US 2011/0294737 A1
(43) Pub. Date: Dec. 1, 2011

(54) TERMINALLY-DIFFERENTIATED ANUCLEATE PLATELET PROGENY GENERATION

(76) Inventors: Hansjorg Schwertz, Salt Lake City, UT (US); Robert C. Blaylock, Salt Lake City, UT (US); Larry W. Kraiss, Salt Lake City, UT (US); Guy A. Zimmerman, Salt Lake City, UT (US); Andrew S. Weyrich, Salt Lake City, UT (US)

(21) Appl. No.: 13/122,886

(22) PCT Filed: Oct. 7, 2009
(86) PCT No.: PCT/US09/05497

§ 371 Date: May 27, 2011

Related U.S. Application Data
(60) Provisional application No. 61/195,558, filed on Oct. 7, 2008.

(51) Int. Cl. A61K 38/02; A61K 31/7072; A61K 31/551; A61K 31/496; A61K 31/536; A61P 7/00; A61K 31/203; A61K 31/708; A61K 31/7068; A61K 31/513; A61K 31/52; C12N 5/078; A61K 31/505

(52) U.S. Cl. 514/13.5; 435/325; 435/391; 514/50; 514/220; 514/253.09; 514/230.5; 514/272; 514/559; 514/45; 514/49; 514/274; 514/263.4

(57) ABSTRACT

Platelets are induced to proliferate, form extensions and produce daughter cells by various methods, including culturing platelets under thrombocytopenic conditions. Expansion of platelet cell numbers increases the storage life of platelets. Modulation of RT activity can be used to produce new daughter platelets. Therefore, the invention provides a new therapeutic use for RT inhibitors that can now be used for treatment of thrombocytopenia and related disorders. Likewise, application of soluble protein factor that may be secreted and/or released by platelets cultured under thrombocytopenic conditions may also be used as a therapeutic agent to increase platelet numbers.

### STATEMENT REGARDING FEDERALLY SPONSORED RESEARCH OR DEVELOPMENT

This work was funded by NIH grants HL066277, HL044525 and HL075507, Western Affiliate American Heart post-doctoral fellowship (0625098Y) and NSF grants (DMR-0602684 and DBI-0649865) and the Harvard MRSEC (DMR-0213805).

### TECHNICAL FIELD

This invention relates to the field of biotechnology, more particularly to progeny cells generated from anucleate platelet cells, methods of inducing production of progeny cells, methods of using progeny cells for the treatment of diseases and methods of expanding platelet cell populations.

### BACKGROUND

The references discussed herein are provided solely for the purpose of describing the field relating to the invention. Nothing herein is to be construed as an admission that the inventors are not entitled to antedate a disclosure by virtue of prior invention. Furthermore, citation of any document herein is not an admission that the document is prior art, or considered material to patentability of any claim herein, and any statement regarding the content or date of any document is based on the information available to the application at the time of filing and does not constitute an affirmation or admission that the statement is correct.

The renewal of terminally-differentiated eukaryotic, such as red blood cells, platelets and polymorphonuclear leukocytes, is carried out by bone marrow hematopoietic progenitors. In the case of platelets, these cells are released from the cytoplasm of parental megakaryocytes and enter the circulation without a nucleus²⁻⁴. Because of their short lifespan (˜9-11 days), the average adult must produce approximately 1×10¹¹ new platelets per day to maintain normal platelet counts under steady state conditions⁵. The number of platelets far exceeds the number of megakaryocytes, which comprise less than 0.1% of the cells in normal bone marrow³. Nevertheless, the current dogma is that the final step of platelet formation occurs when megakaryocytes extend proplatelets through bone marrow sinusoids and shear stress from blood flow prunes these protrusions into single platelets^{2, 4}. There has been no evidence that individual platelets continue to generate additional platelets once they enter the circulation. A common feature of terminally-differentiated hematopoietic cells is that they are typically arrested in the G₀ state and as a consequence, do not produce progeny³¹.

Platelet disorders typically involve an abnormal number of platelets and/or abnormal functioning of the platelets where the disorder affects blood clotting in the subject. For example, platelet cell numbers can drop to dangerously low levels in diseases such as anemia and in subjects being treated with chemotherapy. Such diseases and treatments typically require infusion of platelet cells from another source. Because platelet cells are short lived, there has been a need in the field to either artificially produce platelet cells or to extend the storage times for platelet cells.

In the past, for example, there have been attempts to generate platelets from embryonic stem cell lines. But these methods have a number of difficulties and problems that prevent their use.

Current platelet infusion typically uses either platelets pooled from random donors or single donor apheresis platelets, both of which can be stored up to 5 days at room temperature. Longer storage times have been hampered by bacterial outgrowth and sepsis resulting from bacterial growth. Likewise, storage of platelets at lower temperatures results in the platelets being rapidly cleared from the subject's blood system following transfusion. Therefore, platelet preparations have a very short life span and have to be used or thrown away within a very short period of time.

Stimulation or enhancement of platelet production using thrombopoiesis stimulating factors has been previously described in, for example, U.S. Pat. Nos. 5,571,686; 5,593,666; 5,178,856; 5,087,448; 5,032,396; 5,498,698; 5,498,599; and 5,326,558. There is no suggestion in the prior art, however, that platelets themselves can be stimulated or induced to increase platelet and/or proplatelet production.

Although there is no direct evidence that platelets divide, recent studies have identified unexpected functions of platelets that are under discrete molecular control ^{1, 33, 34}, thus, platelets are far more sophisticated than previously considered ³⁵. Cell division is accompanied by increases in protein synthesis in nucleated cells and the present inventors and others have shown that platelets retain the capacity to process pre-mRNA ^{1, 6, 36, 37} and translate mRNA into protein ³⁸⁻⁴². There is also recent evidence that platelets continue to synthesize protein for days when they are stored ex vivo⁴³. These studies suggest that platelets are dynamic cells that continue to alter their phenotype as they circulate in the blood.

### SUMMARY OF THE INVENTION

The invention relates to the ability to induce proliferation in platelet cells. The invention also relates to methods of culturing platelets under thrombocytopenic conditions to induce production of newly extended cell bodies, which separate to produce new daughter platelets. This expansion method may be used to increase the storage life of platelets. Data presented herein shows that the new daughter platelets are structurally and functionally similar to their parents.

The method can be applied to aged platelets after several days of storage to generate new daughter platelets, and has applications in blood banking and other such industries. The method can further be applied to the proliferation of platelets in plasma and in whole cultured blood, thereby providing advantages to transfusion technologies and blood storage.

The invention also provides a method of inducing expansion and/or production of daughter platelets by modulation of RT (reverse transcriptase) activity. Thus, the invention provides a new therapeutic use for RT inhibitors in promoting platelet production and/or expansion. The invention also provides a method of modulating expansion of platelet cells by administration of retinoic acid compounds and other agonists/ligands of the retinoid X receptors. In an exemplary embodiment, treatment of platelet cultures with 9-cis retinoic acid results in decreased RT activity, and induces production of daughter platelets.

The invention provides new daughter platelet cells and methods of generating new platelet cells that may be used for the treatment of thrombocytopenia and related disorders.

The invention also provides a method of inducing expansion of platelets by application of a soluble protein factor that is secreted and/or released by platelets cultured under conditions conducive to expansion and production of daughter platelets. In an exemplary embodiment, the protein factor is secreted into the culture media by the platelets, is soluble and is between 10-30 kDa. These experiments were performed using size-exclusion spin columns as described further below.

This protein factor may be used as a therapeutic agent for thrombocytopenia and thrombocytosis disorders. It may also be used as a biomarker.

In an exemplary embodiment, the invention provides a method of treating thrombocytopenic conditions. For example, platelets from a subject may be expanded by culturing the platelets under conditions that induce production of progeny platelets and the expanded number of platelets then reintroduced into the subject.

In another exemplary embodiment, the present invention provides a method of expanding a platelet population by diluting the platelets with a culture media that is formulated to stimulate platelet expansion and to be administered to a subject. Thus, a platelet population may be expanded without requiring purification of the platelets from the media prior to use in a subject.

The invention also relates to the manufacture of a medicament comprising an RT inhibitor, including retinoic acid compounds and other agonists/ligands of the retinoid X receptor, for the treatment of thrombocytopenia and thrombocytosis disorders.

### BRIEF DESCRIPTION OF THE DRAWINGS

FIGS. 1A-1I illustrate that freshly-isolated or ex vivo aged platelets cultured under mildly thrombocytopenic conditions extend projections with distinct cell bodies. FIG. 1A illustrates the localization of actin (stained with phalloidin) and sialic acid stained with WGA) in human platelets that were fixed immediately after isolation (Baseline, the three panels on the left) or after 6 hours in suspension (Cultured, the three panels on the right). The bottom row displays corresponding transmission images. As described herein, discoid platelets extended projections with bulbar areas resembling cell bodies. FIG. 1A is representative of over 20 independent experiments. Scale bars=5 μm. FIG. 1B depicts freshly-isolated platelets that were diluted to a concentration of 1×10⁵ per mm³ (low, middle panel) or 1×10⁶ per mm³ (high, right hand panel), cultured for 6 hours and stained for actin. The panels display a representative example of twenty studies. In FIG. 1C, the bar graph indicates the percentage of extended cells with at least two cell bodies/μl (mean±SEM; n=13). Single asterisk: p<0.05, low platelet condition compared to baseline. The cell body formation process was not due to fusion of metabolically active platelets with one another, as shown in FIG. 1D, and occurred in individual platelets that were confined in drops of culture media suspended in oil and stabilized with inert surfactant. Separate samples of platelets were labeled with dyes of two different colors and subsequently incubated together for 6 hours. Labeled platelets (blue or green) independently developed extensions with cell bodies (see arrows). The left and rights panels of FIG. 1D display two independent experiments which are representative of three. In FIGS. 1E-1G, platelets that were confined in microdrops developed extended projections with distinct cell bodies. FIGS. 1E and 1F illustrate that platelets confined in microdrops extend projections with distinct cell bodies. Platelets were loaded into “parked” microdrops as described further herein. In FIG. 1E, platelets were loaded in droplets and examined at baseline or after 6 hours (cultured). The thin arrows point to single platelets (baseline) or the same platelet that formed two distinct cell bodies after 6 hours (cultured). The thick arrows point to unique landmarks for each position in the microfluidic device. In FIG. 1F, sequential images of platelets using low-resolution wide-field microscopy are shown. The arrows highlight the location of the platelets within each drop during the course of the experiment. After 6 hours, the single platelets formed two distinct cell bodies (see black arrows, far right panels. Distance between the white brackets (see far left panels) is 50 μm. FIG. 1G shows representative examples of freshly-isolated (top row) or ex vivo aged platelets (day 4, bottom row) immediately after they were confined in drops (A, F) or 6 hours after they were cultured in drops (B-E and G-J). As shown in FIG. 1G, cultured platelets developed extensions with distinct cell bodies, or in some cases, remained round (panels E and J). These images are representative of the typical morphology of platelets of over 100 platelets analyzed in this manner. FIGS. 1H and 1I are panels which display examples of freshly-isolated (FIG. 1H) or ex vivo aged (FIG. 1I) platelets that are representative of platelets that were considered to have two or more cell bodies, as further referenced in FIG. 1D.

FIGS. 2A-2C show that platelets that develop new cell bodies display typical platelet features. In FIG. 2A, cultured platelets with two or more cell bodies express critical biomarkers and spread on extracellular matrix. Freshly-isolated platelets were cultured (6 hrs) alone or in the presence of thrombin (0.01 U/μl) (far right panels of FIG. 2A). From left to right in the top row, the immune-staining identifies α_{IIb}β₃, P-selectin, β-tubulin, control IgG, respiring mitochondria (Mitotracker), or sialic acid (WGA). Corresponding transmission images are shown in the bottom row of FIG. 2A. Scale bars=5 μm. FIG. 2A is representative of three independent experiments. FIG. 2B demonstrates that newly-formed platelets possess granules and organelles. Thin section TEMs of representative platelets are shown at baseline (I, VI, VII) and after six hours in culture (IIV, VIII). The black scale bar represents 500 nm. Low magnification (15000×) shows typical round and elliptical thin sectioned platelets at baseline (I) compared to more elongated platelets observed after 6 hours in culture (II). Representative ultrastructural changes in platelets after 6 hours in culture are shown in panels III-V where the platelets are connected by cytoplasm of various lengths. Multiple alpha granules (red arrows) are observed at both platelet body ends (III-V) and occasionally in the connecting region (III). A constricted region resembling a cleavage furrow is noted along the long shaft of a cultured platelet (V with inset, magnification 80000×, scale bar represents 100 nm). Magnification is 25000× (III), and 30000× (IV, V). Baseline platelets with alpha granules (red arrows) and microtubules (blue arrows) in cross section (VI; magnification 30000×) and transverse section (VII; magnification 40000×). An example of how platelet diameters were measured by TEM (VIII; magnification 30000×) to demonstrate that cell diameters were significantly (p<0.05) increased in cultured platelets when compared to freshly-isolated platelets (data not shown). Microtubules in cross section were also observed at ends of the cultured platelets (blue arrows; VIII). Mature and budding granules are present in cell bodies of extended platelets and their shafts lack well-defined membranes as shown in FIG. 2C. The top panel of FIG. 2C shows a TEM image of a cultured platelet, which was layered onto a flat surface so the entire cell could be observed in one section. Three distinct dilated areas suggestive of cell bodies are observed in the center panel. The center panel of FIG. 2C is a higher magnification of one of the bulbar regions (see right rectangle, top panel). The white arrows highlight a budding granule. The bottom panel of FIG. 2C is a higher magnification of a shaft between two cell bodies (see left rectangle, top panel of FIG. 2C) that lacks a well-defined membrane (brackets). Scale bar in top panel=1 μm. Scale bars in center and bottom panels=250 nm. FIG. 2C is representative of three independent experiments.

FIGS. 3A-D illustrate that newly-formed cell bodies are functional. The bar graph of FIG. 3A illustrates that cultured platelets respond to activating stimuli. The bar graph depicts P-selectin and PAC-1 surface expression (% positive cells) as assessed by flow cytometry in freshly-isolated (0 h) or cultured (6 h) platelets with or without thrombin (Thr) stimulation for 15 minutes. The data are compared to isotype-matched control antibodies (IgG, IgM). FIG. 3B shows that cultured platelets that develop processes spread and divide on extracellular matrix. Platelets were left in suspension culture for 6 hours and subsequently placed on immobilized fibrinogen. As shown in the sequential images (I-XII) of FIG. 3B, an extended platelet adheres, spreads, and forms two distinct cell bodies that eventually separate from one another (see red dashed lines). Scale bar=5 μm. FIG. 3B is representative of four independent experiments. FIGS. 3C and 3D show that newly-formed platelets respond to activating stimuli. Platelets were cultured for 6 hours and subsequently treated with vehicle, ADP (2 μM) or thrombin (0.005 U/ml). After 60 seconds, the platelets were fixed in solution, the permeabilization step was skipped, and then the cells were co-immunostained for P-selectin and sialic acids, as shown in FIG. 3C, or Annexin V and actin, as shown in FIG. 3D. FIGS. 3C and 3D are representative of three independent experiments. Scale bars=10 μm.

FIGS. 4A-4E show that cultured platelets increase in biomass and accumulate protein. FIG. 4A shows that cultured platelets increase in diameter, volume and biomass. The four bar graphs, from left to right, show the mean±SEM for diameter (I), volume (II), thickness (III), and biomass (IV) of freshly-isolated versus cultured platelets. Single asterisk: p<0.05 versus baseline (I-III) or 0 h (IV). The bar graph of FIG. 4B shows that cultured platelets accumulate protein. The graph shows the mean±SEM for total protein concentration of freshly-isolated (baseline) versus cultured platelets. Single asterisk: p<0.05 versus baseline. In FIG. 4C, the top two panels show protein expression patterns for freshly-isolated (baseline) versus cultured (6 h) platelets. These two-dimensional gels, which are tilted in a third dimension to more effectively display the peak intensity and height of individual proteins, are representative of five independent experiments. The pie chart of FIG. 4C categorizes the protein expression patterns in freshly-isolated versus cultured platelets. The categories are labeled as newly expressed (spots identified in cultured platelets that were not present at baseline), upregulated (spots that were increased in cultured platelets when compared to baseline), down-regulated (spots that were decreased in cultured platelets when compared to baseline), or no change (spots that remained constant between cultured and baseline platelets). The percentages in the pie chart are the average of five independent experiments. FIG. 4D shows freshly isolated platelets which were pre-incubated with puromycin (top right panel), a global inhibitor of translation, or vehicle (top middle panel) for 2 hr and subsequently cultured in the presence of an azido amino acid analog of methionine (Met AA analog). The platelets were fixed after 6 hours and incorporation of the methionine analog into protein was visualized in each cell. The bottom panels depict the same cells labeled with WGA. Unlabeled platelets fixed at baseline (left panels) were used to control for background fluorescence. These panels are representative of three independent experiments. The figure shows that changes in protein expression are, in part, due to de novo protein synthesis, as shown by the incorporation of an azido modified methionine analog into platelets that develop new cell bodies. Inhibition of Met AA incorporation by puromycin resulted in reduced formation of new cell bodies. In FIG. 4E, protein expression for mitofilin, P-selectin, actin, and GAPDH in platelets at baseline or cultured for 6 hours in the presence or absence of puromycin are shown. These immunoblots are representative of three independent experiments.

FIGS. 5A-D show that stored platelets develop new cell bodies and increase in number. In FIG. 5A, ex vivo aged platelets are shown to form new cell bodies. Ex vivo aged (1 or 4 days) platelets were resuspended in M199 medium and immediately fixed (baseline) or cultured in suspension for 6 hours. In FIG. 5A, the panels display a representative example of one study where the platelets were stained for actin. Scale bars=10 μm. In FIG. 5B, the bar graph indicates the number of ex vivo aged platelets with at least two cell bodies/μl (mean±SEM; n=4). Single asterisk: p<0.05, cultured versus baseline. FIG. 5C illustrates that ex vivo aged platelets that develop two or more cell bodies express critical biomarkers. Ex vivo aged platelets (day 4) were resuspended in culture medium for 6 hours in the presence (far right panels) or absence of thrombin. From left to right in the top row, the red stain identifies α_{IIb}β3, P-selectin, β-tubulin, control IgG, respiring mitochondria (Mitotracker), or sialic acids (WGA). Corresponding transmission images are shown in the bottom row. Scale bars=5 μm. This figure is representative of three independent experiments. In FIG. 5D, stored platelets are shown to increase in number. Platelets were stored under standard blood bank conditions and platelet counts, as well as mean platelet volumes (MPV), were determined. The left graph shows the platelet count before (Day 0) and after (Day 5) storage (mean±SEM; n=10). The right panel displays the MPV obtained from platelets used for the counting studies. Single asterisk: p<0.05, Day 0 versus Day 5 for both panels.

FIG. 6 shows that an intact microtubular network is required for progeny formation. The panels of FIG. 6 display baseline platelets (left column) and cultured platelets (right three columns) that were left alone or treated with reagents that disrupt microtubular function (i.e., nocodazole or taxol). The top row shows specific immunostaining for β-tubulin (magenta) with corresponding transmission images displayed in the bottom row. Treatment of the platelets with the nocodazole or taxol results in formation of tear-drop like platelets that lack new cell bodies. This figure is representative of three independent experiments. Scale bars=10 μm.

FIG. 7 shows that platelets cultured in fresh human plasma form new cell bodies. FIG. 7 shows freshly-isolated (baseline) platelets (left panel) and platelets that were cultured at 1×10⁸ ml in anti-coagulated plasma (ACD) at 37° C. (cultured) (right panel). The platelets were stained with α-tubulin (green) and P-selectin (red). As shown in the right panel, P-selectin coalesced to the middle of cell bodies and α-tubulin was located along the rims of the newly-formed platelets. This figure is representative of three independent experiments. Scale bars=10 μm.

FIG. 8 illustrates by a dot blot graph that large platelets are rare in freshly-isolated cell preparations. The dot blot indicates TEM diameter measurements along the longest axis of freshly-isolated platelets (baseline). The closed circles show a homogenous cell population that contains very few large platelets.

FIGS. 9A-D. Cultured platelets replicate their mitochondrial DNA and increase in number. Cultured platelets were incubated with or without BrdU and the cells were prepared for flow cytometry (FIG. 9A) or immunocytochemistry (FIG. 9B). The bar graph in FIG. 9A represents the mean±SEM (n=3) of the percentage of platelets that incorporated BrdU. FIG. 9B shows BrdU localization in cultured platelets. Controls for each of these figures included omission of the anti-BrdU antibody (No primary Ab) or quenching of the antibody with BrdU (Quench). Scale bars=5 μm. FIG. 9C shows the results from platelets that were incubated with (lane 1) or without (lane 2) α³²P-dTTP for 6 hours. Radiolabelled thymidine was incorporated into mitochondrial DNA (arrow). FIG. 9D illustrates platelet numbers at baseline and after 6 hours in culture. The bar graph displays the percent increase in cell numbers over baseline that occurred in cultured platelets. The lines in each bar represent the mean±SEM of five independent experiments. Single asterisk: p<0.05, between baseline and stored platelet conditions.

FIGS. 10A-C illustrate the effects of inhibition of endogenous reverse transcriptase activity on cultured platelets. FIG. 10A depicts platelet lysates that were treated with vehicle (DMSO) or nevirapine and then incubated with MS2 phage RNA. Reverse transcription of MS2 phage RNA was carried out as previously described²². The lanes are as follows: 1: DMSO; 2-4: nevirapine (100, 500, and 750 μM, respectively); 5: no RNA; 6: lysis buffer only; 7: no cell lysate; 8: no RNA and no cell lysate; 9: commercial RT; 10: no reverse primer in RT reaction; and 11: negative PCR. FIGS. 10B and 10C denote platelets (1×10⁵ per mm³) that were left untreated or were treated with DMSO or nevirapine (750 μM) and subsequently cultured for 6 hours. FIG. 10B shows microscopy panels that display representative examples of cultured platelets in the absence (untreated) or presence of nevirapine (scale bars=10 μm)), with nevirapine treatment resulting in an increase in the number of projections with distinct cell bodies extending from cultured platelets. FIG. 10C is a bar graph that displays the percent increase in the number of platelets with at least two bulbs in treated versus untreated platelets (mean±SEM; n=8). Single asterisk: p<0.05, nevirapine compared to untreated or DMSO treated platelets, which were morphologically similar to untreated platelets (data not shown).

FIGS. 11A-C illustrate inhibition of endogenous reverse transcriptase activity in platelets in response to exposure to 9-cis retinoic acid, which also increases the number of projections with distinct cell bodies that extend from cultured platelets. FIGS. 11A and 11B show platelets (1×10⁵ per mm³) that were left untreated or were treated with DMSO or 9-cis retinoic acid (1 and 10 μM) and subsequently cultured for 6 hours. FIG. HA illustrates microscopy panels that display representative examples of cultured platelets in the absence (untreated) or presence of 9-cis retinoic acid (scale bars=10 μm). FIG. 11B is a bar graph displaying the percent increase in the number of platelets with at least two bulbs in treated versus untreated platelets (mean±SEM; n=3). Single asterisk: p<0.05. Platelets treated with 9-cis retinoic acid are compared to untreated or DMSO treated platelets, which were morphologically similar to untreated platelets (data not shown). FIG. 11C depicts reverse transcriptase activity in platelet lysates that were treated with vehicle (DMSO) or 9-cis retinoic acid and then incubated with MS2 phage RNA to detect the activity of reverse transcriptase. Reverse transcription of MS2 phage RNA was carried out as previously described²². The lanes are as follows: 1: DMSO; 2-4: 9-cis retinoic acid (0.1, 1, and 10 μM, respectively); 5: no RNA; 6: lysis buffer only; 7: no cell lysate; 8: no RNA and no cell lysate; 9: commercial RT; 10: no reverse primer in RT reaction; and 11: negative PCR.

FIG. 12 is a bar graph that illustrates daughter platelet production in response to culture density and either platelet culture supernatant or size exclusion fractions derived from cultured platelet supernatant. The bar graph displays the percent increase in the number of platelets with at least two bulbs in the different treatment groups. The bars are as follows: 1: baseline; 2: platelets cultured at 1×10⁶ per mm³; 3: platelets cultured at 1×10⁵ per mm³; 4: platelets cultured at 1×10⁶ per mm³ with the total supernatant from platelets cultured at 1×10⁵ per mm³; 5: platelets cultured at 1×10⁵ per mm³ with the total supernatant from platelets cultured at 1×10⁶ per mm³; 6: platelets cultured at 1×10⁶ per mm³ with the supernatant filtrate from the kDa size exclusion column from platelets cultured at 1×10⁵ per mm³; 7: platelets cultured at 1×10⁶ per mm³ with the supernatant retentate from the 10 kDa size exclusion column from platelets cultured at 1×10⁵ per mm³; 8: platelets cultured at 1×10⁶ per mm³ with the supernatant filtrate from the 30 kDa size exclusion column from platelets cultured at 1×10⁵ per mm³; and, 9: platelets cultured at 1×10⁶ per mm³ with the supernatant retentate from the 30 kDa size exclusion column from platelets cultured at 1×10⁵ per mm³.

### DETAILED DESCRIPTION OF THE INVENTION

As used herein, “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of.”

As used herein and in the appended claims, the singular forms, for example, “a”, “an”, and “the,” include the plural, unless the context clearly dictates otherwise. For example, reference to “a platelet” includes a plurality of such platelets, and reference to a “protein” is a reference to a plurality of similar proteins, and equivalents thereof.

As used herein, “about” means reasonably close to, approximately or a little more or less than the stated number or amount.

As used herein, “blood” means whole blood or any fraction thereof, for example plasma, platelets, and/or a concentrated suspension of cells.

As used herein, “diagnosis” or “diagnostic” means a prediction of the type of disease or condition from a set of marker values and/or patient symptoms.

As used herein, “disordered coagulation” includes, but is not limited to, thromboembolic disease, intravascular thrombosis, microvascular platelet thrombosis, venous thromboembolism, deep vein thrombosis, disseminated intravascular coagulation (DIC), coronary artery disease, fibrinolysis and/or sepsis.

As used herein, “prognosis” or “prognostic,” means to predict disease progression at a future point in time from one or more indicator values.

As used herein, “sample” means any sample of biological material derived from a subject, such as, but not limited to, blood, plasma, mucus, biopsy specimens and fluid, which has been removed from the body of the subject. The sample which is tested according to the method of the present invention may be tested directly or indirectly and may require some form of treatment prior to testing. For example, a blood sample may require one or more separation steps prior to testing. Further, to the extent that the biological sample is not in liquid form (for example, it may be a solid, semi-solid or a dehydrated liquid sample); it may require the addition of a reagent, such as a buffer, to mobilize the sample.

As used herein, “subject” means a mammal, including, but not limited to, a human, horse, bovine, dog or cat.

As used herein, “platelets” or “platelet cells” means a preparation enriched for platelet cells, microparticles, or a combination thereof.

As used herein, “proplatelets” means any structural form of a megakaryocyte or its fragments, such as cytoplasmically-linked platelet-like particles, that could result in platelet formation. The structural forms include, but are not limited to, cells with long cytoplasmic extensions, projections or pseudopodia that contain swellings encompassing platelet bodies in various stages of formation, such as nodules, blebs and the like.

As used herein, “promoting platelet expansion” means a process that induces or advances the production of extensions or projections (e.g., ranging from 10-200 μm), multiple cell body-like bulges, bulbar regions, segmented constrictions or short tails in a population of generally round platelets.

As used herein, a “blood platelet disorder” means a condition or disorder caused by blood platelet dysfunction or insufficiency, or an over supply of blood platelets. Blood platelet disorders include, but are not limited to, thrombocytopenia, thrombocythernia and thrombocytopathy.

As used herein, “thrombocytopenia” means a condition characterized by a relatively low production of platelets or low platelet count and includes, but is not limited to, increased breakdown of platelets in the bloodstream (intravascular thrombocytopenia) and increased breakdown of platelets in the spleen or liver (extravascular thrombocytopenia). Examples of thrombocytopenia conditions include, but are not limited to, aplastic anemia, bone marrow cancer, bone marrow infections, bone marrow transplants, malignant infiltration, HIV and other viral infections, leukemia, cardiopulmonary by-pass, sepsis, antibody-mediated platelet destruction, genetic disorders (e.g., May-Hegglin, Sebastian Syndrome, Fechtner Syndrome), pregnancy (mild), hemolytic uremic syndrome, immune thrombocytopenic purpura (ITP), drug-induced immune thrombocytopenia, drug-induced non-immune thrombocytopenia, thrombotic thrombocytopenic purpura, neonatal thrombocytopenia, dilutional thrombocytopenia, disseminated intravascular coagulation (DIC), idiopathic thrombocythernia, chronic myelogenous leukemia, myeloid metaplasia and hypersplenism.

As used herein, “thrombocytopathy” means a blood platelet disorder characterized by a relatively high or low platelet function, regardless of the platelet count, which may be within a normal range. Examples of thrombocytopathic disorders in which the platelet function is low include Mediterranean thrombocytopathy, von Willebrand's disease and idiopathic (immune) thrombocytopenic purpura. Low platelet function thrombocytopathic conditions can also be associated with, or result from, HIV infections, dru-induced or hereditary storage pool disorders, uremia and myelodysplastic disorders or thrombolytic therapy. Exemplary thrombocytopathic disorders in which the platelet function is high include thrombocythemia.

As used herein a “carrier” or a “vehicle” means a material suitable for formulation of a composition that is to be administered to a subject and includes any such material known in the art which is non-toxic and does not interact with other components of the composition in a deleterious manner.

The dosage regimen for treating and/or preventing blood platelet disorders with the compounds, compositions, or methods of the invention, such as by administration of an RT inhibitor, is selected in accordance with a variety of factors, including the age, weight, sex, diet and medical condition of the subject; the severity of the disease; the route of administration; pharmacological considerations such as the activity, efficacy, pharmacokinetic and toxicology profiles of the particular compound used; whether a drug delivery system is used and whether the compound is administered as part of a drug combination. Likewise, a dosage regimen for in vitro expansion of platelets is selected in accordance with a variety of factors, including the culture media, temperature, CO₂ levels, the activation state of the expansion factor, the presence or absence of other proliferation enhancers, the time and duration of administration and other such conditions and factors.

The compounds and compositions of the present invention can be administered by any available and effective delivery system, including, but not limited to, orally, bucally, parenterally, by inhalation spray, by topical application, by injection, transdermally or rectally, in dosage unit formulations containing conventional nontoxic pharmaceutically acceptable carriers, adjuvants and/or vehicles. Injection includes subcutaneous injections, intravenous injections, intramuscular injections, intrasternal injections, and infusion techniques.

Platelets may be used by children and adults having diseases such as leukemia, aplastic anemia, cancer, chemotherapy and other diseases of the blood. Because of a malfunction in the bone marrow, chemotherapy treatment or other such reasons, these subjects are unable to produce platelets, have an insufficient amount of platelets and/or have platelets lacking a desired function. These subjects typically need platelet transfusions. While an infusion of fresh platelets may not cure a disease, they provide patients with the time necessary for the treatment to work or for the subject to begin producing his or her own platelets again. Without an infusion of fresh, healthy donor platelets, the recovery and prognosis for many of these patients would be uncertain.

While human platelets were used to illustrate the invention, mouse platelets also produce newly-formed cell bodies when they are cultured in vitro, which demonstrates that the response is conserved (data not shown). As a result, it is believed that the invention is applicable to all mammals and/or platelets from any source, such as horse, cow, pig, dog or cat.

In an exemplary embodiment, the invention provides a method of generating newly-formed daughter platelets by culturing platelets under conditions that mimic mild thrombocytopenia.

In another exemplary embodiment, the invention provides a method of expanding a population of platelets by adding culture media to the platelets or adding platelets to culture media. Optionally, a RT inhibitor may be added to the platelets and/or culture media. The cultured platelets may then be kept in a temperature controlled environment at a temperature of about 37° C. until progeny platelets are produced. The platelets may then be separated from the culture media and used for the treatment of a subject or stored for possible future use in the treatment of a subject.

The present invention provides a method where anucleate platelets can be induced to produce functional progeny. Anucleate platelets spawn new cell bodies that display typical functional characteristics. The formation of new cell bodies is associated with an increase in platelet biomass, protein synthetic events, and total intracellular protein. The progeny formation also leads to a significant increase in platelet numbers, which may have immediate clinical impact for transfusion medicine.

These results are unexpected because platelets were thought to be fully differentiated and arrested in a G₀ state³², and therefore incapable of any type of cellular fission. If thrombopoiesis continues in the blood stream, it may explain how scant numbers of bone marrow megakaryocytes maintain trillions of platelets in the circulation. Given that the process is under regulatory control, the invention provides new therapies for the treatment of thrombocytopenia and the expansion of stored human platelets.

For example, non-activated platelet cells may be cultured to expand the platelet population, which provides a solution to problems associated with plasma storage. For example, platelet cells that have already been stored in a platelet bag for up to four or five days may be removed from the platelet bag and cultured to induce production of new platelets, which can then be stored for another 4 to 5 days prior to use. Likewise, platelets may be isolated from a subject suffering from leukemia or any other condition that may require or benefit from the addition of platelets. Following platelet isolation, the platelets may be cultured to expand the number of platelets present in the sample, and the expanded population of platelets reintroduced into the subject.

Inhibition of Reverse Transcriptase in platelet cells stimulates the generation of daughter platelets. Therefore, the invention provides a method of using RT inhibitors. RT inhibitors include, but are not limited to, non-nucleoside RT inhibitors (NNRTI), such as Nevirapine, Delavirdine, Evafirenz, Etravirine and MK-076, and nucleoside RT inhibitors (NRTI), such as AZT, ddI, ddC, d4T, 3TC, ABC and FTC. Furthermore, RT activity is inhibited in response to treatment with retinoic acid compounds and other agonists/ligands of the platelet retinoid X and retinoid acid receptor families, such as 9-cis retinoic acid, which directly or indirectly inhibits RT activity in cultured platelet cells.

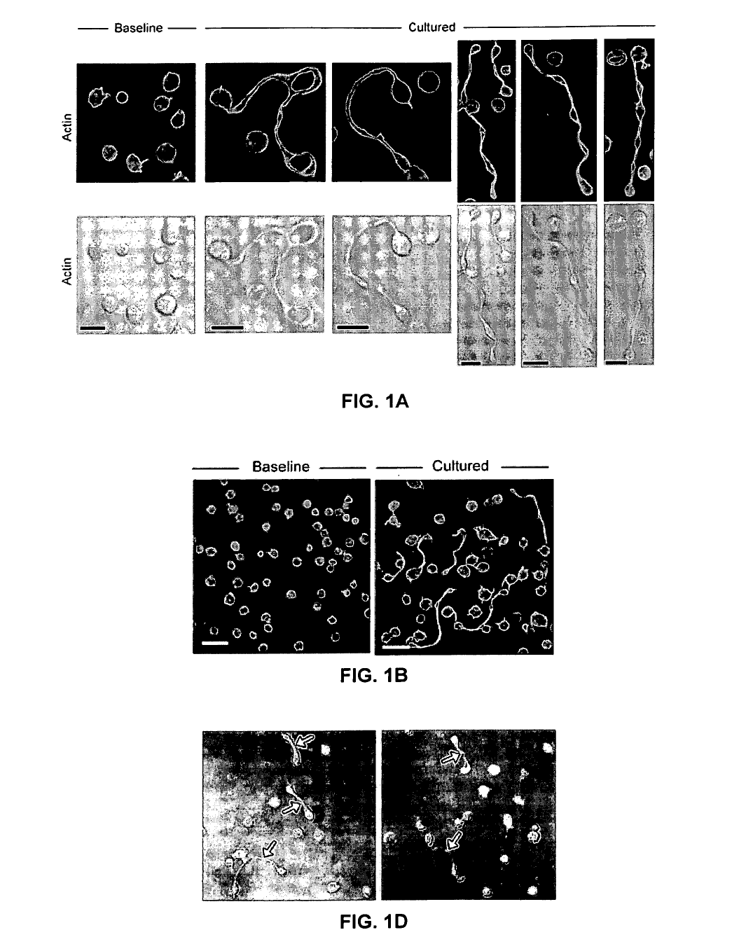

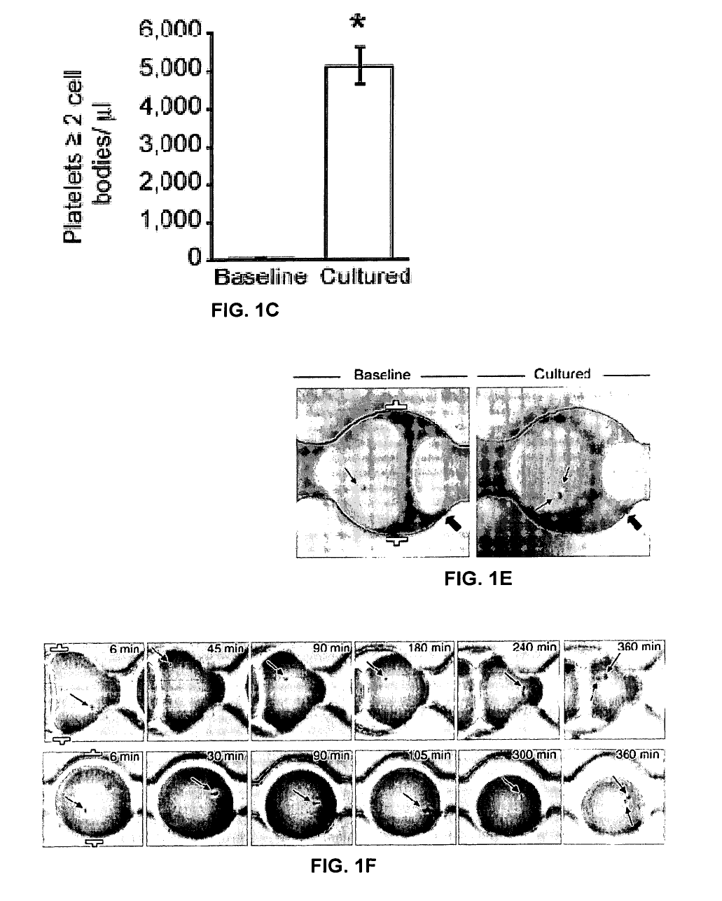

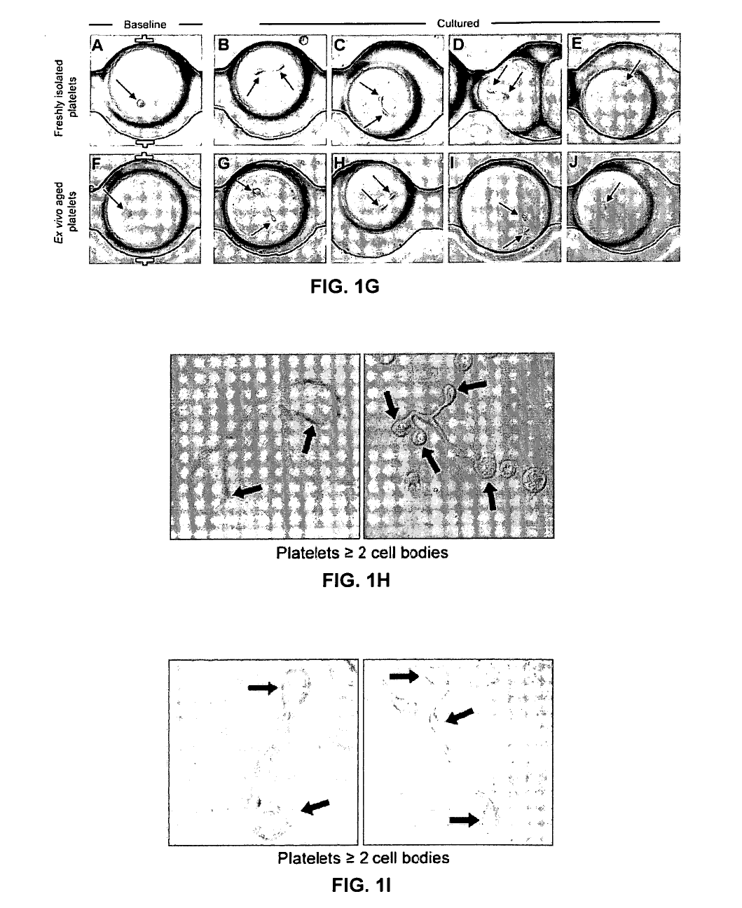

The present invention provides conditions that allow anucleate platelets to spawn daughter cells. Freshly-isolated platelets were used in the initial studies. Contaminating leukocytes were removed from the preparations followed by an isolation step that yields purified platelets with a typical quiescent morphology^{1, 6}. As shown in FIG. 1A (also see left panel of FIG. 1B), baseline platelets were discoid with no structural signs of activation. In contrast, after 6 hours in culture media, many of the round platelets extended projections that resembled proplatelet processes previously described in megakaryocytes³ (FIGS. 1A and 1B). The extensions varied in number and length (i.e., ranging from 10-200 μm), and typically contained multiple cell body-like bulges (FIGS. 1A, 1B). In addition, polymerized actin rimmed the newly-formed cell bodies and sialic acid-rich structures, which co-localized with P-selectin (data not shown), were concentrated in the core of each bulbar region. The cell bodies were separated from one another by segmented constrictions that were often bent and, in some cases, bifurcated into additional processes. In addition, platelets with short tails were commonly observed; suggesting that they were either transitioning into extended cells or, conversely, recently separated from longer processes. Ring-like platelets of different sizes were also frequently observed in the cultured milieu (FIG. 1A), similar to observations of platelets in freshly-isolated plasma.

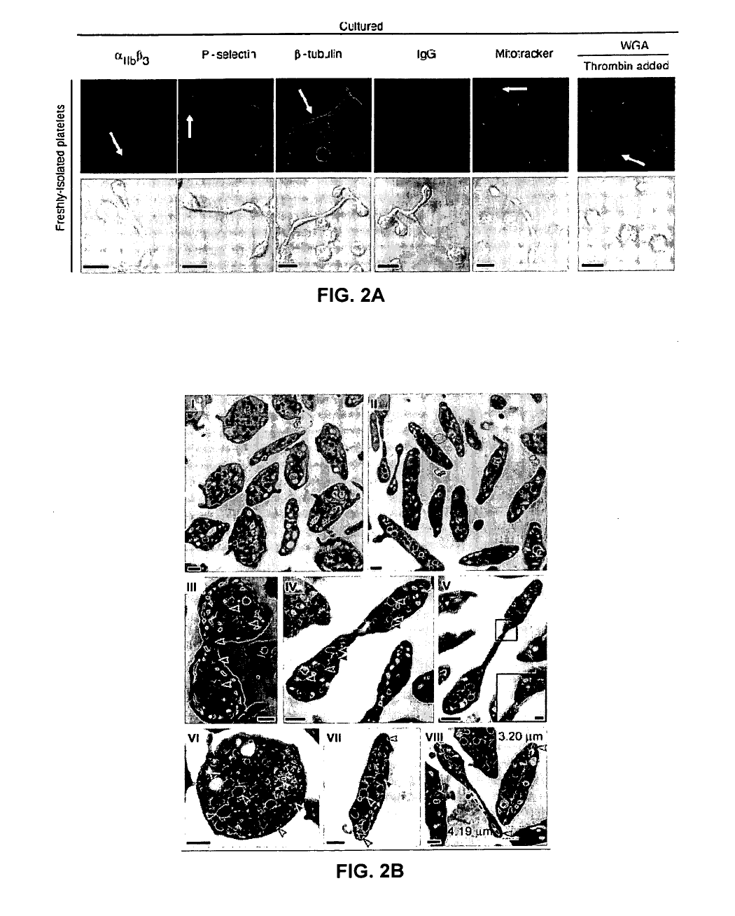

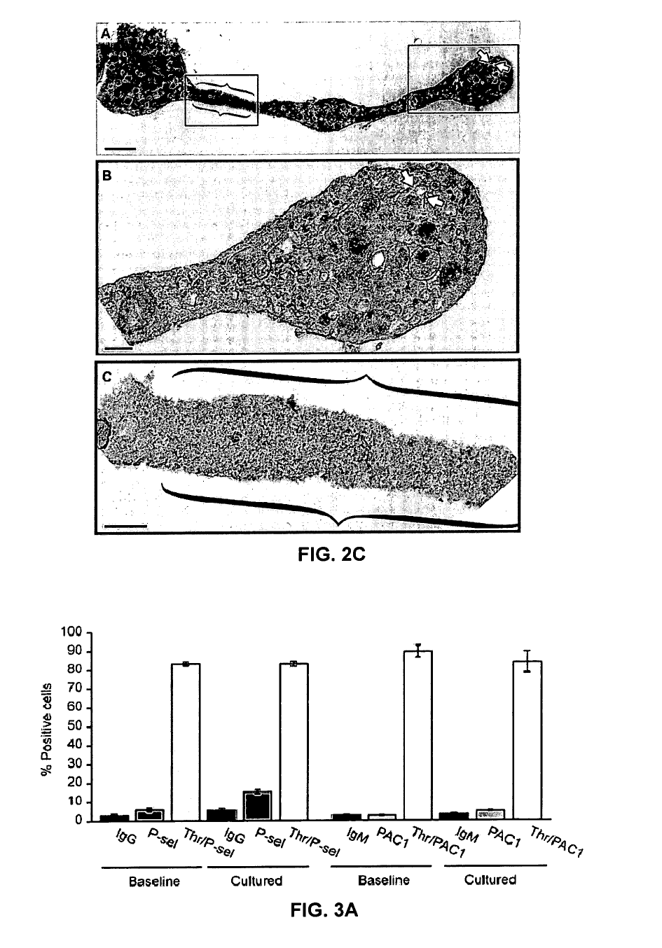

This process was not due to fusion of metabolically active platelets with one another (FIG. 1D) and occurred in individual platelets that were confined in drops of culture media suspended in oil and stabilized with inert surfactant. FIG. 1E and FIG. 2C show examples of individual platelets that formed additional cell bodies after a 6 hour incubation period. Additionally, sequential tracking of the cells demonstrated that single platelets underwent morphologic changes at different times during the incubation period and in numerous cases developed two distinct cell bodies (FIG. 1F).

Furthermore, no nuclei were detected in any of the cells with specific stains (data not shown) indicating that the extended cells were not atypical megakaryocytes, which have been observed in peripheral blood⁶. Formation of extensions with cell bodies was dependent on the number of platelets in the culture. Platelets incubated under conditions equivalent to thrombocytosis (1×10⁶ per mm³) did not generate extensions with cell bodies (FIG. 1B). Conversely, platelets resuspended at a concentration of 1×10⁵ per mm³, which is equivalent to mild thrombocytopenia, readily produced extensions with multiple cell bodies (FIG. 1B). Platelets only generated processes when they were placed in non-adherent, suspension cultures (FIGS. 1A and 1B). Adherence to plastic, extracellular matrices or stimulation with agonists that induced the release of granular contents blocked this process, indicating that platelet activation inhibits the development of these morphological changes (data not shown; also see far right panel of FIG. 2A).

Several groups have detected barbell-shaped cells and beaded platelet processes in the systemic circulation^{4, 7, 9, 10}. By inference, all of these morphologic variants were thought to be bone marrow megakaryocyte-derived progeny that continue to morph in the circulation to engender individual, “young” platelets^{7, 11}. Therefore, experiments were conducted to determine if aged platelets generate extensions.

Human platelets were harvested from single donors by apheresis and stored in plasma at 20-24° C. under constant agitation in an FDA approved platelet bag. At designated times; platelets were removed under sterile conditions from the storage bags, gently washed, and diluted in culture medium to 1×10⁵ cells per mm³. At baseline, platelets stored for 1 or 4 days looked similar to freshly-isolated platelets with no structural signs of activation (FIG. 1D, data not shown). By contrast, numerous extensions with defined cell bodies developed after 6 hours in culture (FIGS. 1D and 1E). These results demonstrate that platelets can generate extensions with cell bodies for days after they have been harvested.

Low-resolution, wide-field microscopy was used to sequentially track the morphology of platelets in microdrops over 6 hours. Serial images of the same platelet were obtained over a 6 hour period. FIG. 1F shows two representative images of single platelets that formed two distinct cell bodies during the incubation period. In both cases, single platelets readily moved within the drop and elongated over time. By 6 hours, two distinct cell bodies were observed.

To unequivocally demonstrate that a single platelet can form bead-like daughter structures, individual round platelets were confined in drops using microfluidic parking chambers. Freshly-isolated and aged platelets were loaded into drops of culture media, suspended in oil and stabilized with inert surfactant. In the first set of studies, the morphology of cells immediately after loading into drops was compared with the morphology of cells cultured in drops for 6 hours. FIG. 1G depicts representative examples for both conditions. Platelets, whether they were freshly-isolated or aged, reproducibly exhibited a bi-concave and discoid shape immediately after they were loaded in the drops (FIG. 1G, baseline). In contrast, after 6 hours in the drops, many of the platelets generated extensions with obvious dilatations, usually at the ends (FIG. 1G, cultured). Some of the freshly-isolated or stored platelets remained round during the 6 hour incubation period (FIG. 1G, far right panels). In all cases, z-series analysis demonstrated that the platelets readily moved within the drops without adhering to the surfactant-coated interfaces over the time course of the experiment (data not shown).

The newly formed extensions and cell bodies express essential platelet proteins and functional mitochondria. An integrand unique to megakaryocytes and platelets that controls homonymic aggregation^{12, 13}, αIIbβ3, was observed on the surface of freshly-isolated or aged platelet extensions and newly-formed cell bodies (FIG. 2A). Likewise, P-selectin, a key constituent of platelet α-granules that regulates interactions of platelets with leukocytes¹⁴, coalesced to the center of newly-formed cell bodies (FIGS. 2A and 2B). Surface expression of P-selectin, however, did not increase during the 6 hour culture period (data not shown). β-tubulin was observed in the shafts of each extension and coiled around the rim of the cell bodies (FIG. 2A). Mitofilin, a mitochondrial membrane protein that controls cristae morphology¹⁵, was detected in mitochondria (data not shown), and dyes that recognize respiring mitochondria were readily observed in cell bodies, and occasionally in shafts, suggesting that organelles traffic through these microtubular-rich regions to reach bulbar regions of the cell (FIG. 2A). In contrast, thrombin-stimulated platelets did not develop new cells bodies (FIG. 2A and data not shown). Polymerized actin rimmed the newly-formed cell bodies in these extended platelets and sialic acid-rich structures, which co-localized with P-selectin (data not shown), were concentrated in the core of each bulbar region (FIG. 2A). Incubation of the platelets with cytocholasin D, a potent inhibitor of actin polymerization, inhibited this morphologic transition indicating that actin filaments are required to build the projections (data not shown).

Transmission electron microscopic (TEM) analyses of the processes confirmed that the newly-formed cell bodies were packed with granules and other organelles (FIG. 2B). Microtubules and microfilaments were commonly observed in the shafts between the cell bodies, similar to the organization of microtubules in constricted areas of proplatelets extending from megakaryocytes⁴⁴. In some regions, however, the shafts lacked defined cellular membranes (FIG. 2B, 2C). The absence of distinct membranes in segmented constrictions is suggestive of cellular pinching and resembles constricted furrows in dividing mitotic cells¹⁷ and proplatelet extensions⁴⁴. Budding organelles were also observed at the ultrastructural level (data not shown; FIG. 2C, middle panel).

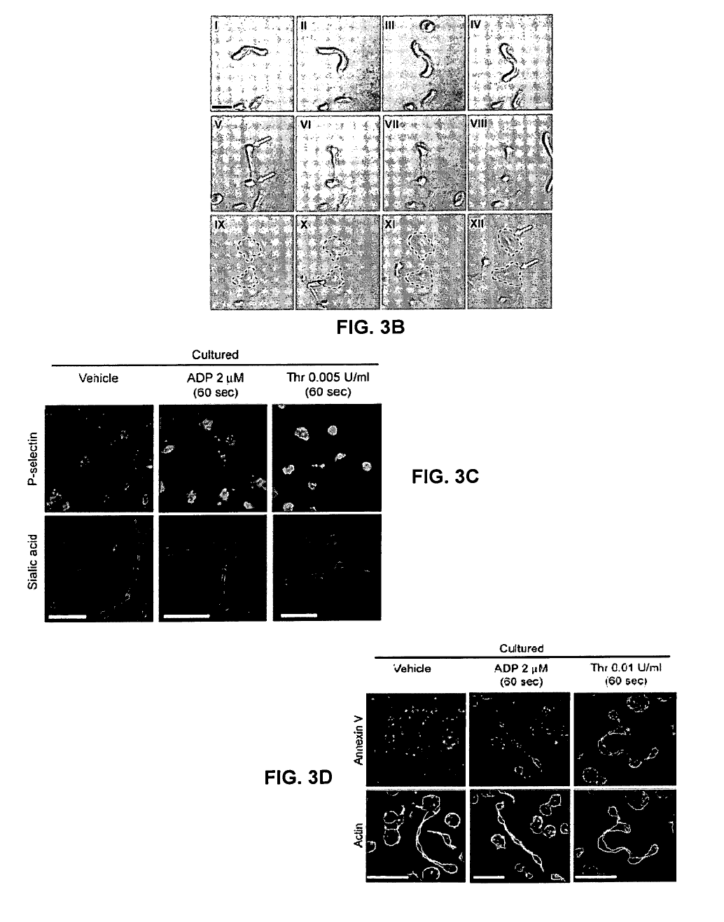

Newly-formed platelets are metabolically active and functional (FIG. 3A). It was observed that agonist-induced expression of surface P-selectin and PAC-1 binding, an α_{IIb}β₃-dependent response, did not wane over the 6 hour culture period (FIG. 3A). Extended platelets adhere to and spread on immobilized fibrinogen, a response that relies on conformational changes in α_{IIb}β₃ integrins¹⁸. Real-time studies confirmed that once extended platelets form in suspension cultures, they can adhere to and spread on immobilized fibrinogen (FIG. 3B) and, in some cases, physically separate from one another (FIG. 2D; data not shown). These studies indicate that the newly-formed cell bodies are functional. Many of these binary platelets (i.e., 2 platelets linked together by a stalk) separated into two individual cells or, in rare cases, retracted back to single platelets as they spread on the extracellular matrix (FIG. 3B). Surface P-selectin expression was markedly increased in platelets that developed new cell bodies when they were activated with low concentrations of thrombin or ADP for 1 minute (FIG. 3C). Similarly, newly-formed platelets displayed increased annexin V on their surface in response to cellular activation (FIG. 3D). When multi-bodied platelets were activated with high concentrations of agonist or for prolonged periods of time (i.e., 2-5 minutes), thin shafts between the cell bodies disappeared indicating that the platelets separated from one another (data not shown).

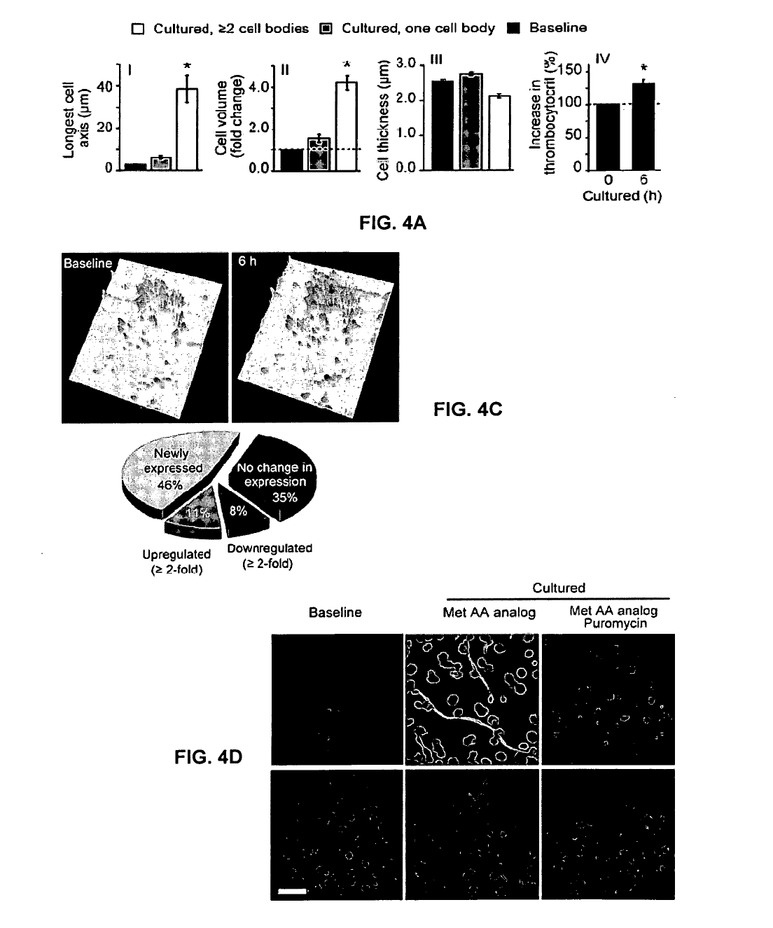

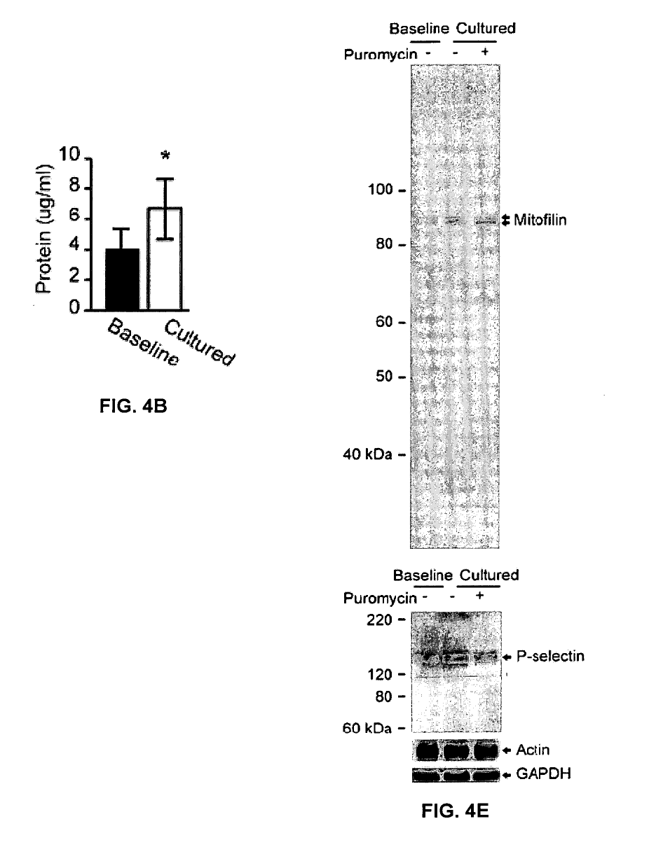

As nucleated cells prepare for cell division they increase in size. To determine if similar changes occur in progeny forming platelets, platelets were fixed in suspension and gently layered them on a flat surface to assess the entire cell in a single plane. Using this methodological procedure, it was observed that the maximal diameter (FIG. 4A.I) and volume (FIG. 4A.II) of platelets increased (p<0.05) during the culture period whether the cells were actively forming new cell bodies or not. In contrast, the thickness of platelets decreased slightly, in large part, because the shafts that connect the cell bodies were very thin (FIG. 4A.III and data not shown).

Dividing cells typically increase their biosynthetic activity in preparation for cytokinesis, which involves redistribution of cytoplasm, organelles and cell membranes into daughter cells ¹. Similarly, it was observed that the biomass (FIG. 4A.IV) and intracellular protein content (FIG. 4B) of platelets increased significantly (p<0.05) as they produced new cells. Separation of the intracellular proteins by 2-D gel electrophoresis also demonstrated an increase in total protein as well as a substantial shift in intracellular protein expression patterns (FIG. 4C). Increased protein expression was, in part, due to protein synthesis because an azido modified methionine analog readily incorporated into platelets that develop new cell bodies (FIG. 4D). Methionine incorporation was blocked by the translational inhibitor puromycin, which also reduced the development of new cell bodies (FIG. 4D). Puromycin also reduced the accumulation of mitofilin, a mitochondrial specific protein and P-selectin, an α-granular protein that has recently been shown to be under regulatory control in circulating platelets ⁴⁵ (FIG. 4E). In contrast, β-actin and GAPDH did not show any change in expression pattern due to the culture conditions (FIG. 4E).

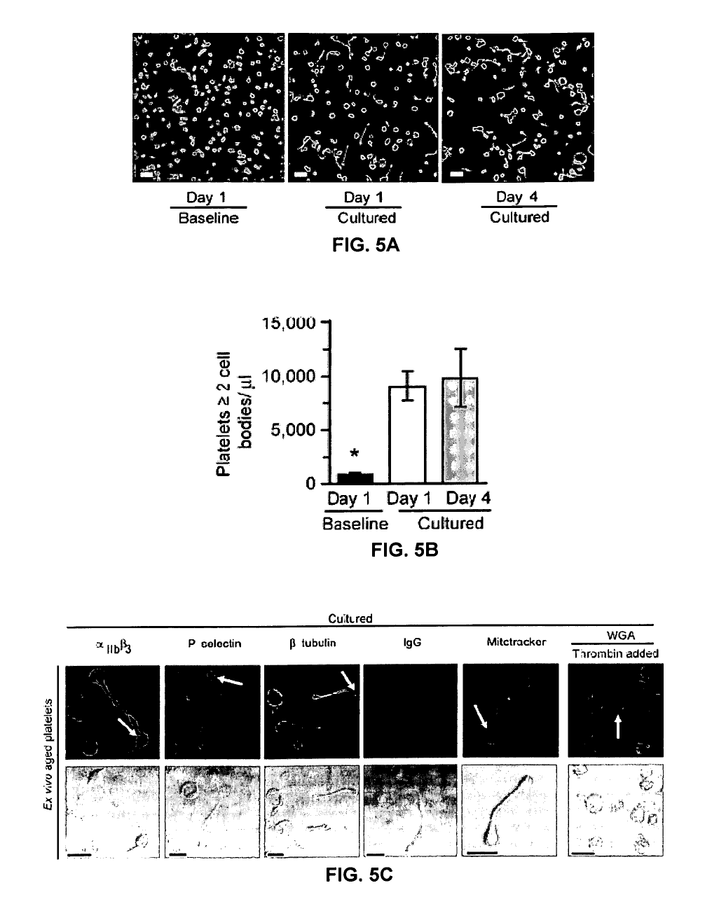

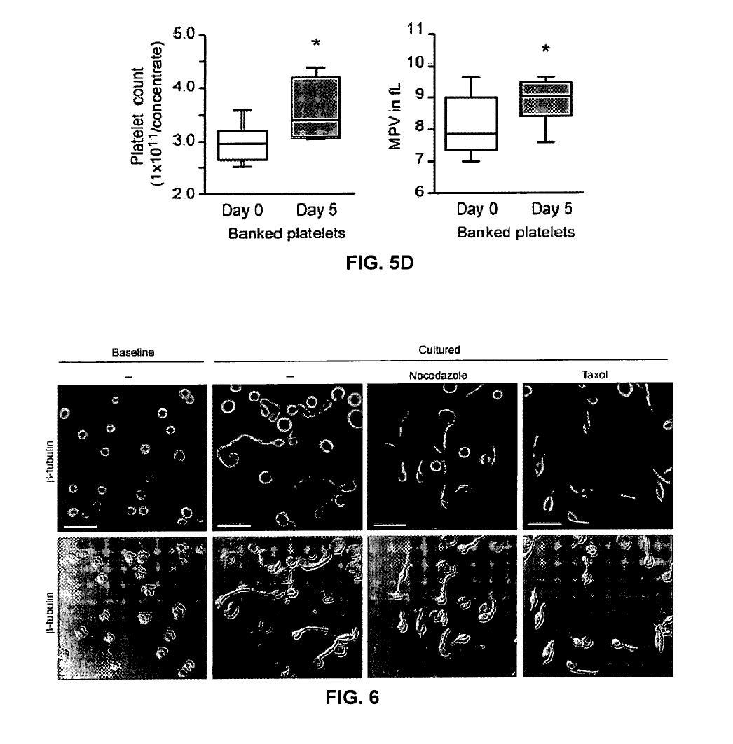

Ex vivo aged platelets are also capable of developing new cell bodies and increase in number. Long projections of megakaryocytes have been demonstrated in bone marrow sinusoids ⁴ and similar types of projections have been identified in freshly-isolated plasma ^{4, 7, 9, 10}. To determine if mature platelets have the capacity to generate new cell bodies and increase in number outside of the bone marrow milieu, platelets were removed from the circulation and were aged ex vivo. Human platelets were harvested from single donors by apheresis, which, by design, filters out the majority of leukocytes. In the first set of studies, apheresed platelets were stored in plasma at 20-24° C. under constant agitation in an FDA approved platelet bag. After 1 or 4 days of storage, the platelets were removed under sterile conditions, gently washed, and resuspended in culture medium. Baseline platelets (i.e., time 0) were characteristically discoid (data not shown) and similar to freshly-isolated platelets, and the ex vivo aged platelets readily formed new cell bodies (FIGS. 5A, 5B and 1G). The new cell bodies expressed integrin α_{IIb}, P-selectin, β-tubulin, and contained respiring mitochondria (FIG. 5C).

Experiments were undertaken to determine if platelet counts increased in cells that were stored under standard blood bank conditions. It was observed that platelets from 9 of 10 donors increased in number, which was accompanied by a significant increase in mean platelet volume (FIG. 5D). The average increase was 21.1±5.1% (p<0.01 versus day 0) with a maximal increase of 46.6% (i.e., day 0=2.955×10¹¹ platelets/concentrate vs. day 5=4.369×10¹¹ platelets/concentrate). The number of freshly-isolated platelets also increased significantly after only 6 hours in suspension culture (data not shown). It was also observed that platelets form extensions in whole blood. Anti-coagulated (ACD) whole blood cultured at 37° C. was shown to enable platelets to form extensions with new cell bodies. To achieve these results, whole blood was first spun down to remove part of the platelet-rich plasma to reduce the platelet numbers in the whole blood preparation. The volume removed was replaced by media M199. The table below shows that platelets ready for transfusion in FDA-approved transfusion bags increase in number while stored under FDA conditions:

| Concentrate | Platelet count increase in |
|---|---|
| No. | % over 5 days storage |
| 013710 | 12.3 |
| 014510 | 13.7 |
| 796104 | 46.6 |
| 793106 | 46.2 |
| 917103 | 3.5 |
| 918106 | 20.1 |
| 916107 | 33.2 |
| 915108 | 5 |
| 229101 | −2.2 |
| 750103 | 32.8 |
| Mean | 21.12 |
| SEM | 5.6 |

Collectively, the data from these studies reveal that a terminally differentiated cell, i.e., the platelet, can produce progeny. Similar to cell division in nucleated cells, progeny formation is associated with increases in protein synthesis and mitochondrial DNA replication (unpublished observations). Unlike nucleated cells, however, platelets do not uniformly split into two daughter cells but instead produce multiple cell bodies that are packed with granular constituents and organelles (FIG. 2B). The newly-formed platelets possess typical functional activity that includes the ability to adhere, spread and express surface adhesion molecules in response to agonist stimulation.

The mechanisms that regulate progeny formation in platelets are incomplete. However, it is notable that thrombopoietin, a key regulator of bone marrow-dependent thrombopoiesis, has no effect on progeny formation (data not shown). Additionally, exposure of platelets to thrombin (far right panels of FIGS. 2A and 5C), gram-negative E. coli (data not shown), or adherence to extracellular matrix, blocks the development of new cell bodies. This indicates that platelet activation is a negative regulator of progeny formation. Further, an intact microtubular network facilitates the formation of new cell bodies. Platelets treated with taxol, which stabilizes microtubules, or nocodazol, which impairs polymerization of microtubules, do not form progeny. Instead, platelets display a “teardrop-like” morphology, possessing short tails that are devoid of new cell bodies (FIG. 6). Microtubules are essential for cell division and dynamic microtubules accommodate shape changes in platelets ⁴⁶ and regulate the development of proplatelets that extend from the cytoplasm of megakaryocytes ⁴⁷. This indicates that megakaryocytes and mature platelets use similar structural mechanisms to form bulbar extensions and that progeny formation may be an extension of proplatelet evolution in the circulation ^{4, 44}.

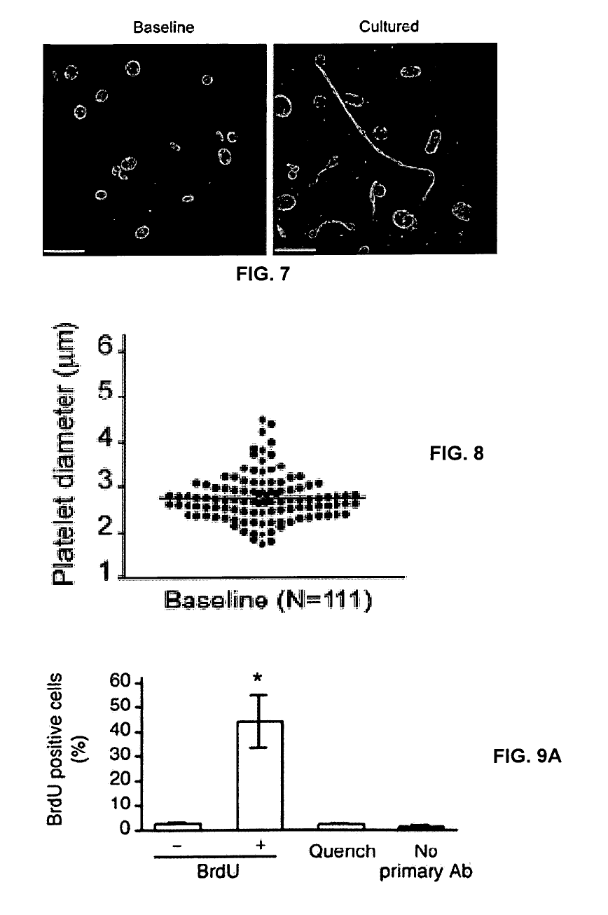

Strings of platelets with multiple cell bodies are commonly observed in whole blood⁷. Whether or not they are directly shed from the cytoplasm of megakaryocytes or morphed products of circulating platelets is not known. However, platelets with multiple cell bodies are more frequently observed in response to acute thrombocytopenia, before megakaryocytes increase in size, ploidy and number^{9, 10, 48}. Platelets also form new cell bodies when they are resuspended in plasma (FIG. 7); this suggests that mature platelets may be capable of spawning daughter cells in the bloodstream. While it is currently believed that progeny formation occurs more readily in young versus old platelets, it has been clearly demonstrated that ex vivo aged platelets develop new cell bodies, suggesting that the process may not be restricted to young platelets.

The biologic function of progeny formation by platelets is not fully determined as yet, but it is believed that platelets produce daughter cells that are more thrombogenic or programmed for cell death. Alternatively, it is believed possible that thrombopoiesis continues in the bloodstream, providing an explanation as to how scant numbers of bone marrow megakaryocytes maintain trillions of platelets in the circulation. The “fission-like” process demonstrated in the current data challenges the paradigm that terminally-differentiated eukaryotic cells are incapable of expanding their population. From a clinical perspective, the ability to feasibly increase the number of human platelets during blood or platelet storage provides new possibilities for transfusion medicine and provide basis for the development of new treatment regimens for thrombocytopenia.

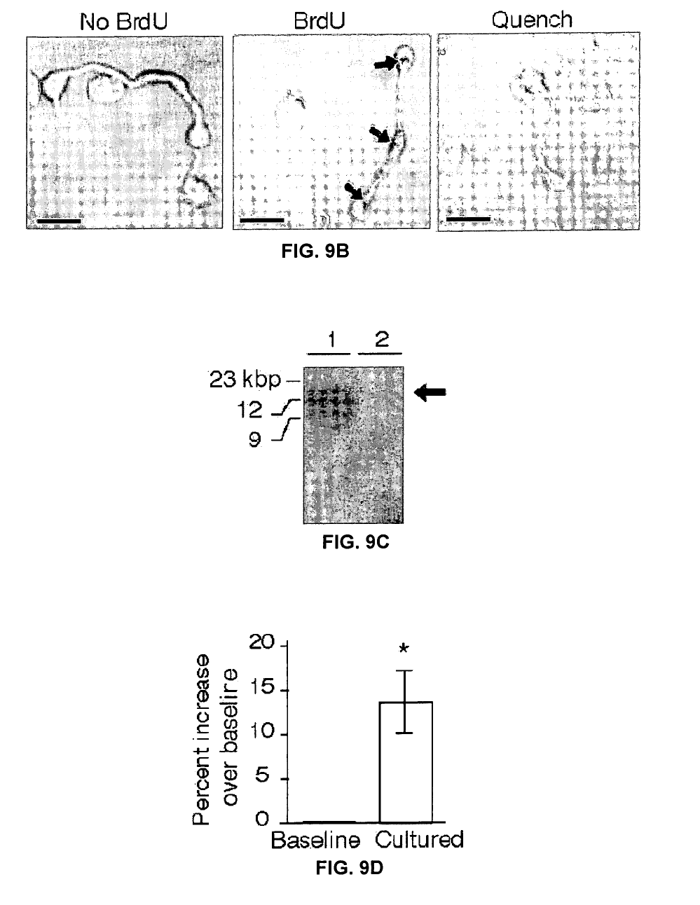

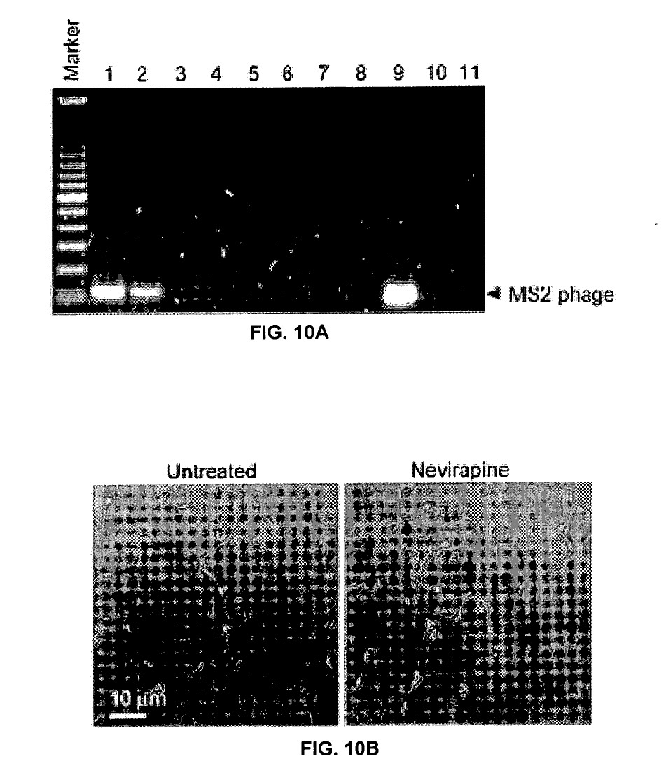

The results illustrated in FIGS. 1A-1, 2A-C and 5A-B demonstrate that freshly-isolated or aged platelets generate extensions with distinct cell bodies. Therefore, human platelets have the capacity to elongate and divide, even though they lack nuclei. Cell proliferation in nucleated cells is associated with an increase in the replication rate of mitochondrial DNA (mtDNA), which is important for maintaining mitochondrial function and meeting the energy demands of the cell¹⁹. Therefore, 5-bromo-2′-deoxyuridine (BrdU) was incubated with platelet cells to determine if it would be incorporated into newly synthesized mtDNA. The amount of BrdU incorporation was significantly increased in platelets after 6 hours in culture (FIG. 9A) and localized to mitochondria in the newly-formed cell bodies and microtubular-rich shafts of platelets (FIG. 9B). Radiolabelled thymidine was also detected in mitochondrial DNA (mtDNA) (FIG. 9 4C).

Next, platelets were counted at baseline or after 6 hours in culture to determine if mtDNA replication is associated with increases in cell number. Cultured platelets increased in number between 6% and 22%, with an average increase of approximately 15,000 newly-formed platelets for every 100,000 cells (i.e., about 13%) (FIG. 9D). Increases in platelet numbers of this magnitude are considerable given that trillions of platelets circulate in the bloodstream. Consistent with this increase, cultured platelets expressed more protein for mitofilin, P-selectin and integrin α_{IIb}β₃ and inhibition of protein synthesis prevented each of these critical genes from increasing in quantity (data not shown). These results are consistent with the hypothesis that organelle biogenesis occurs as platelets extend and form new cell bodies. In this regard, budding organelles were also routinely observed at the ultrastructural level (FIG. 2C), and after reviewing multiple samples, the number of granules and organelles is believed to be greater in platelets with extensions and multiple cell bodies compared to single platelets.

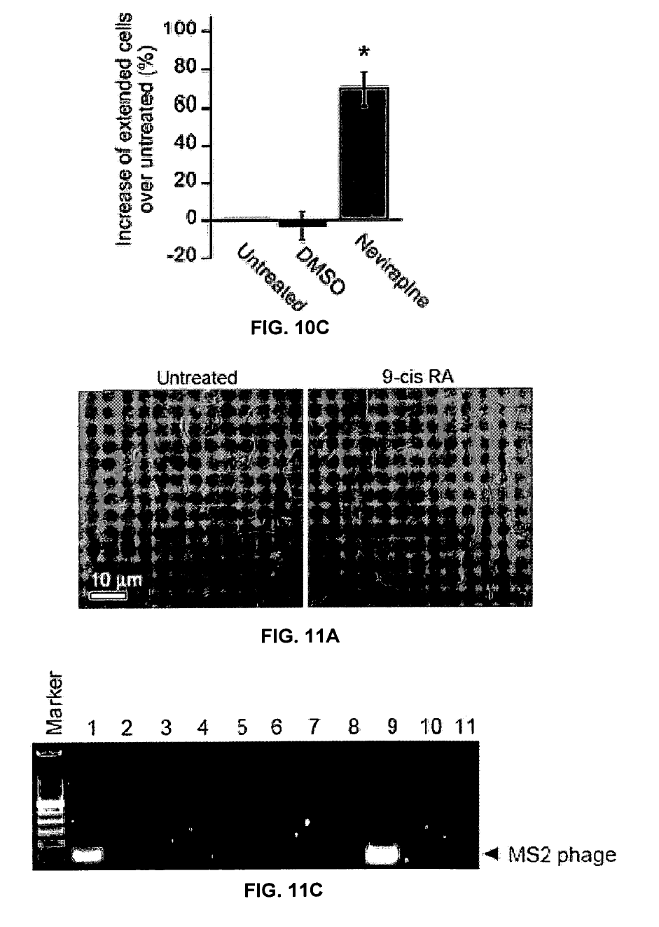

Generation of platelet progeny differs from division in nucleated eukaryotic cells, where one cell produces two daughter cells that have the same genetic make-up as the parent^{20, 21}. From a morphological perspective, the process in platelets is strikingly similar to the formation of proplatelets that develop from the cytoplasm of bone marrow megakaryocytes¹⁶. However, the molecular signals that trigger the response are distinct because thrombopoietin, which modulates nearly all aspects of platelet formation in the bone marrow⁵, has no effect on the development of newly-formed cell bodies that arise from individual platelets (data not shown). Therefore, other molecular pathways are involved in generation of platelet progeny. Endogenous reverse transcriptase (RT) activity is a candidate pathway for regulation of platelet expansion. Inhibition of RT in tumor cells reduces cell growth and division and induces cellular differentiation^{22, 23}. RT activity is present in platelets and is inhibited by the non-nucleoside RT inhibitor nevirapine (FIG. 10A). Inhibition of endogenous RT activity by nevirapine (FIGS. 10B and 10C) or azidothymidine (AZT; data not shown) markedly increased the number of newly-formed cell bodies that extended from cultured platelets. The source of RT activity in platelets is believed to be long interspersed elements-1 (LINE-1), which are robustly expressed in platelets (data not shown). Previous studies have shown that primary non-dividing somatic cells²⁴ possess LINE-1-dependent RT activity and silencing of LINE-1 in cancer cell lines induces cellular differentiation^{22, 25}. The role of RT activity in the generation of platelet progeny indicates that platelets use previously unrecognized pathways to differentiate and/or propagate further in the bloodstream.³¹

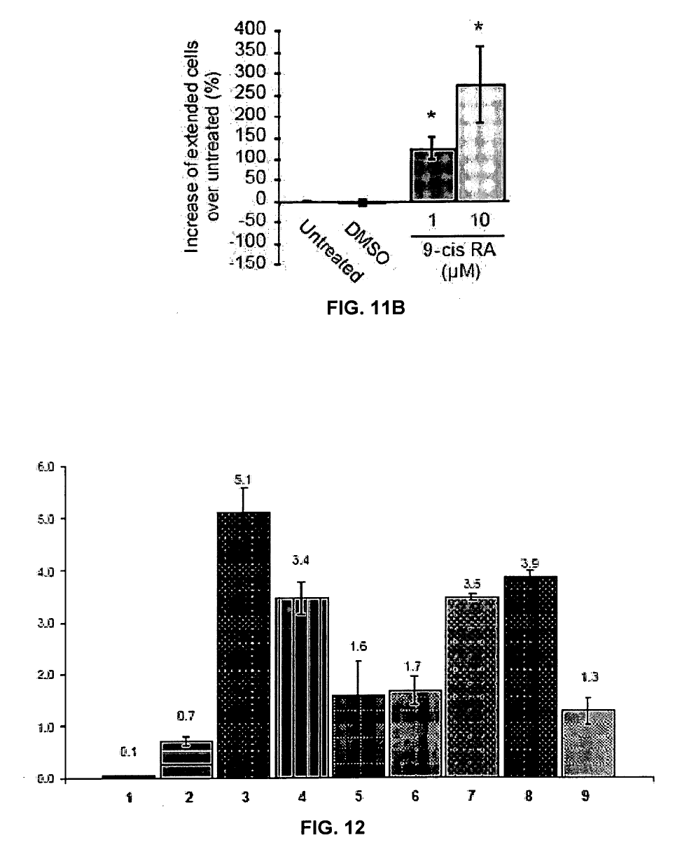

FIGS. 11A-C show that administration of 9-cis retinoic acid (and compounds that modulate retinoid X receptors) induce production of daughter platelets. In particular, administration of 9-cis retinoic acid reduces the level of RT activity in the platelets, which leads to an increase in the production of daughter cells (FIGS. 11A and B). FIG. 11C shows that RT activity is present in platelets and is inhibited by the administration of 9-cis retinoic acid. This shows that administration of 9-cis retinoic acid decreases RT activity in the platelet cells, which increases production of daughter cells.

Further, platelet cells that are capable of producing daughter cells also produce a soluble protein factor sized from between about 10 and about 30 kDa which stimulates the production of daughter cells in other platelet cells. Platelets were cultured under mild thrombocytopenic conditions (1×10⁵ per mm³) or under non-thrombocytopenic conditions (1×10⁶ per mm³). After 2 hours under culture conditions, the platelets were pelleted, and the supernatant from the platelets cultured at 1×10⁶ per mm³ was used to resuspend platelets cultured at 1×10⁵ per mm³ and vice versa. The platelets were then cultured for approximately another 2 hours. In some cases the supernatants from the low concentrated platelets were subject to different size exclusions columns (10 kDa and 30 kDa) and the filtrate or the retentate was added to the high-concentrated platelets.

FIG. 12 illustrates daughter platelet production in response to culture density and either platelet culture supernatant or size exclusion fractions from platelet culture supernatant. Platelets cultured at about 1×10⁶ per mm³ yielded a baseline value for the production of extended cells of about 0.7%. Platelets grown at 1×10⁵ per mm³ (a condition that mimics a mild thrombocytopenic condition) yielded a value for the production of daughters of about 5.1%. Platelets grown at 1×10⁶ per mm³ with the supernatant from cells grown at 1×10⁵ per mm³ yielded a value for the production of daughters of about 3.4%. Platelets grown at 1×10⁵ per mm³ with the supernatant from cells grown at 1×10⁶ per mm³ yielded a value for the production of daughters of about 1.6%. Platelets grown at 1×10⁶ per mm³ with the filtrate from an approximately 10 kDa exclusion membrane yielded a value for the production of daughters of about 1.7%. Platelets grown at 1×10⁶ per mm³ with the retentate from an approximately 10 kDa exclusion membrane yielded a value for the production of daughters of about 3.5%. Platelets grown at 1×10⁶ per mm³ with the filtrate from an approximately 30 kDa exclusion membrane yielded a value for the production of daughters of about 3.9%. Platelets grown at 1×10⁶ per mm³ with the retentate from an approximately 30 kDa exclusion membrane or column yielded a value for the production of daughters of about 1.3%. Hence, it is believed that there is a secreted factor between about 10 kDa and about 30 kDa that may be added to highly concentrated platelets (platelets cultured or stored under non-thrombocytopenic conditions) to induce the production of daughter cells.

This factor (e.g., protein factor) may be used as a therapeutic agent for thrombocytopenia and thrombocytosis disorders. It may also be used as a biomarker, diagnostic and/or prognostic for thrombopenic conditions. It would be possible, therefore, to add the factor directly to isolated platelets in the standard FDA storage bag and increase the platelet number.

### Methods

Platelet Isolation and Culture. Whole blood was centrifuged at 150×g for 20 minutes to obtain platelet-rich plasma (PRP). Residual leukocytes were removed from the PRP by CD45+ bead selection as previously described^{1, 6}. The negatively selected platelets were resuspended in serum-free M199 culture medium at 37° C. in a humidified 5% CO₂ atmosphere. For select studies, platelets were resuspended in fresh human plasma. Because of the rigorous leukocyte depletion step, which applies low shear stress to the cells, platelets with two or more cell bodies were rarely observed in the washed preparations (data not shown). Unless otherwise indicated, the washed platelets were cultured in suspension using round-bottom polypropylene tubes (Becton Dickinson, Franklin Lakes, N.J.). Platelets were suspended at 100,000/μl.

Stored platelets were obtained from the ARUP Blood Transfusion Services at the University of Utah or the Institute of Transfusion Medicine at the University of Greifswald. The apheresed platelets were immediately placed in standardized platelet bags and stored under constant agitation in a climate-controlled chamber (Melco Engineering Corp., Glendale, Calif.) that was maintained between 20-24° C. On day 1 and day 4 (i.e., 24 and 96 hours after apheresis, respectively), samples of the ex vivo aged platelets were removed under sterile conditions, gently washed, and subsequently resuspended as described above.

Apheresed platelets used for the experiments shown in FIG. 5D were obtained from healthy blood donors, who had not taken any medication during the previous 10 days, by the apheresis device ComTec (Fresenius GmbH, Bad Homburg, Germany). Platelet concentrates were rested for 2 hours to allow reconstitution of minor platelet activation. Then a separate bag was aligned to the platelet bag tube by sterile docking to obtain a sample of the platelet concentrate. Platelet count and mean platelet volume were determined using an automated particle counter (Sysmex, Sysmex Japan). To reduce counting errors due to high platelet numbers, samples were diluted 1:4 before measuring using PBS-EDTA 2%. Platelet concentrates were stored under agitation as described above for five days and a second sample was obtained for determination of platelet count and mean platelet volume.

Platelet Morphology and Protein Expression. Freshly-isolated or aged platelets were either fixed immediately to assess baseline morphology, or after 6 hours of suspension culture as described above. In select studies, the cells were treated with thrombin (Sigma, St. Louis, Mo.), nevirapine (AIDS Research and Reference Reagent Program, NIH), or AZT (Sigma). In other select studies, the platelets were treated with ADP (Helena Laboratories, Beaumont, Tex.), nocodazole (Sigma), or taxol (Invitrogen, Eugene, Oreg.) at select time points.

For the studies described in FIG. 2D, platelets were placed in suspension culture and after 6 hours, the cells were incubated in 8-well borosilicate chamberslides that were coated with human fibrinogen (Calbiochem, La Jolla, Calif.) to characterize adherence and spreading by real-time microscopy.

For studies that used fixed platelets, paraformaldehyde (4%) was added directly to the suspension culture as previously described^{1, 6, 26} in order to maintain the native morphology of the cells. The fixed platelets (10,000 total for each sample) were subsequently layered onto coverslips coated with Vectabond™ (Vector Laboratories, Burlingame, Calif.) using a cytospin centrifuge (Shandon Cytospin, Thermo Fisher Scientific, Waltham, Mass.). To determine the number of platelets with extensions and distinct cell bodies, fixed cells were counterstained with Alexa Fluor® 488 phalloidin (A12379; Invitrogen, Eugene, Oreg.), a high-affinity probe for F-actin, and/or an Alexa Fluor® 555 (W32464; Invitrogen) conjugate of wheat germ agglutinin (WGA). Three random fields were captured from each independent experiment and at least 500 total cells, which encompassed individual platelets with two or more distinct cell bodies, were counted (FIG. 1H and FIG. 1I).

For protein localization studies by immunocytochemistry (ICC), fixed cells were stained with antibodies directed against α_{IIb}β₃ and Annexin V (ab34407; Abeam, Cambridge, Mass.), P-selectin (sc-6941; Santa Cruz Biotechnology, Santa Cruz, Calif.), β-tubulin (T-5293; Sigma), or mitofilin (MSM02; MitoSciences, Eugene, Oreg.). Specificity of the staining was confirmed for each antibody with isotype-matched nonimmune IgG. The cells were counterstained with either Alexa Fluor® 488 phalloidin or Alexa Fluor® 555 WGA conjugate as described in the previous paragraph. To assess mitochondrial function, use was made of MitoTracker® Red CM-H2XRos (M7513; Invitrogen), a reduced probe that fluoresces when it enters actively respiring mitochondria. For these studies, MitoTracker® (1 μM) was incubated with the live platelets one hour before the end of the experiment.

For protein expression studies, the freshly-isolated or culture platelets were pelleted, resuspended in equal volumes of reducing buffer, and separated by SDS-page as previously described⁶. Western blotting was subsequently performed for mitofilin, P-selectin, actin (#691001; MP Biomedicals, Solon, Ohio), or GAPDH (Mab374; Millipore, Billerica, Mass.).

For the fusion-based studies in FIG. 1H, platelets from the same donor were incubated either with 1 μM CFDA-SE (Vybrant®CFDA SE Cell Tracer Kit, V12883; Invitrogen, Eugene, Oreg.) or with 1 μM CellTrace Far red DDAO-SE (C34553; Invitrogen) for 15 minutes at 37° C. After this incubation period the cells were mixed together in culture medium at a final concentration of 1×10⁵ per mm³ for 6 hours and subsequently prepared for microscopic analysis as described above.

For the ultrastructural analyses, cultured platelets in suspension were fixed in 2.5% glutaraldehyde/1% paraformaldehyde in cacodylate buffer for 20 minutes or in 2.5% glutaraldehyde in PBS buffer overnight. For most of the studies, platelets were washed with 0.1 M phosphate buffer (pH 7.4), followed by dH₂O by centrifugation at 800×g (10 min). Platelets were then post-fixed with 2% osmium tetroxide (60 min), washed twice with dH₂O, dehydrated by a graded series of acetone concentrations (50%, 70%, 90%, 100%; 2×10 min. each) followed by embedding in Epon. Thin sections were examined with an electron microscope after uranyl acetate and lead citrate staining. While this process optimizes ultrastructural integrity, it has two limitations: first, the centrifugation steps (800×g) have the tendency to break shafts between platelets with multiple cell bodies; second, platelets are randomly oriented in the centrifuged pellet reducing the likelihood that the TEM sections will dissect the entire platelet when it sprouts extensions with multiple cell bodies. Therefore, in select studies (FIG. 2C), platelets were fixed in suspension and gently layered them onto poly-L-lysine-coated (P-1399; Sigma) acylar to orient the cells in a single plane.

For the studies in FIG. 4A (I-III), CFDA-labeled platelets (Vybrant® CFDA SE Cell Tracer Kit, V12883; Invitrogen, Eugene, Oreg.) were fixed at baseline or after 6 hours of culture and then gently layered on a glass surface as described above. Stepwise z-series (0.1 μm slices) were conducted to assess cell thickness. Minimum and maximum diameter as well as cell perimeter was determined (Volocity software Version 4, Improvision Inc., a PerkinElmer company, Waltham, Mass.). Platelet perimeter and thickness was used to calculate cell volume and thrombocytocrits were performed to assess platelet biomass.

For the studies in FIG. 3A, freshly-isolated (0 hr) or cultured (6 h) platelets were activated with thrombin or its vehicle. The cells were subsequently incubated with FITC-conjugated antibodies against P-selectin (#555523, BD Pharmingen, San Diego, Calif.) or PAC-1 (#340535, Becton Dickinson, San Jose, Calif.) and then fixed and analyzed on a 5-color FACScan analyzer (BD, San Jose, Calif.) using the BD software CellQuest. Isotype-matched control samples were used to exclude non-specific antibody binding.

For studies described in FIG. 3B (I-XII), platelets were placed in suspension culture and after 6 hours, the cells were incubated in 8-well borosilicate chamberslides that were coated with human fibrinogen (Calbiochem, La Jolla, Calif.) to characterize adherence and spreading by real-time microscopy.

Microfluidic Device for Single Cell Experiments. Microfluidic devices were fabricated by soft lithography²⁷. Negative photoresist SU-8 2025 (MicroChem, Newton, Mass.) was spin-coated onto clean silicon wafers to a thickness of 25 μm, which defines the channel height, and patterned laterally by exposure to UV light through a transparency photomask (CAD/Art Services, Bandon, Oreg.). Sylgard 184 polydimethysolixane (PDMS) (Dow Corning, Midland, Mich.) was mixed with crosslinker (ratio 10:1), degassed thoroughly, poured onto the photoresist patterns, and cured for at least 1 hour at 65° C. The PDMS replicas were peeled off the wafer and bonded to glass slides after oxygen-plasma activation of both surfaces. The microfluidic channels were coated with aquapel (PPG Industries, Pittsburgh, Pa.) by filling the channels with the solution, per manufacturer's instructions, and subsequently flushing them with air prior to the experiments to improve wetting of the channel walls with fluorinated oil. Polyethylene tubing with an inner diameter of 0.38 mm and an outer diameter of 1.09 mm (Becton Dickinson, Franklin Lakes, N.J.) was used to connect the channels to syringes. Glass and polycarbon syringes were used to load the fluids into the devices. Flow rates were set by computer controlled syringe pumps.

For the time-resolved observation of platelet dynamics, individual cells were confined into drops that were roughly 33 μL in volume. Aqueous drops were fabricated in an inert carrier fluid (FC40 fluorocarbon oil, 3M, St. Paul, Minn.) using the microfluidic devices. To stabilize the drops, a PFPE-PEG block-copolymer surfactant (RainDance Technologies, Lexington, Mass.), at a concentration of 1.8% (w/w), was added to the suspending oil. Prior to the experiments the freshly-isolated or aged platelets were resuspended in fresh media. The concentration of platelets was adjusted to 15,000/μl to achieve an average concentration of one cell per two drops according to the Poisson distribution.

Each microfluidic device combined a flow focusing geometry²⁸ for drop production and a storage area where the drops were kept in place for real-time observation. During storage the drops assumed the shape of an ellipsoid with a height of 25 μm, determined by the channel height, and a diameter of 50 μm or less that was determined by the volume. This setup provided adequate spatial confinement while simultaneously allowing the platelets to move freely within the drops, thereby minimizing inadvertent activation of the cells. Defined locations for the drops in the device allowed evaluation of the same drop over time. Rigorous screens with the surfactant-rich oil were also performed to demonstrate that the oil did not activate the platelets (data not shown). For all of the studies, the platelets were cultured in the drops at 37° C. in a humidified 5% CO₂ atmosphere. The permeability of both the PDMS²⁹ and fluorocarbon carrier oil³⁰ to gas ensures sufficient exchange to maintain the cells at the CO₂ level set by the controlled environment.

Mitochondrial DNA Replication. BrdU labeling and detection for flow cytometry and immunocytochemical analysis was performed according to the manufacturer's instructions (Roche Applied Sciences, Penzberg, Germany). In brief, freshly-isolated platelets were cultured as described above in the presence or absence of BrdU (10 μM). After 6 hours the cells were fixed in suspension, washed, permeabilized with PBS/Triton-X (0.1%), and re-washed. Anti-BrdU working solution was subsequently added to the samples and the cells were incubated for 30 minutes at 37° C. in a humid atmosphere. Controls lacking the anti-BrdU antibody or quenched anti-BrdU antibody were included for each experiment. After additional washes, a sheep-derived anti-mouse-IgG fluorescein-conjugated working solution was added to the samples for 30 minutes at 37° C. in a humid atmosphere. The cells were washed again. Flow cytometry was conducted using a Becton Dickinson (Franklin Lakes, N.J.) FACScan flow cytometer. For the ICC studies, mitochondria were stained with MitoTracker® Red CM-H2XRos (M7513; Invitrogen) one hour before the end of the experiment as described above.

For the radiolabelled thymidine studies, platelets were cultured with or without α-[³²P] dTTP. Mitochondria were subsequently isolated using a manufacturer's kit (89874; Pierce, Rockford, Ill.). A “PureLink Genomic DNA Purification Kit” (K-1810-01; Invitrogen, Eugene, Oreg.) was used to isolate DNA from the mitochondria and the radiolabelled and cold samples were separated by electrophoresis with an agarose gel.

Two-dimensional electrophoresis and protein synthetic studies. Two-dimensional gel electrophoresis was performed as previously described ^{49, 50}. In brief, platelets were lysed in lysis buffer containing 7 M urea, 2 M thiourea, 4% CHAPS, 40 mM TRIS (base) and 2 tablets of a protease inhibitor mix per 20 ml of buffer stock solution (CompleteMini®, Roche, Germany). The final protein concentration of each sample was determined by using the method of Popov et al.⁵¹. Isoelectric focusing (IEF) was performed using the Protean IEF Cell (BioRad, Hercules, Calif.) at a temperature of 20° C. Gel strips (pH 3-10L, GE Healthcare) were rehydrated for 12 hours at 50 V using a buffer containing 7 M urea, 2 M thiourea, 2% CHAPS, 0.5% IPG buffer (pH 3-10L), DTT (2.8 mg/mL), and traces of bromophenol blue. The samples were applied as part of the rehydration solution and lysates were run on an 11 cm strip. For the second dimension (SDS PAGE) IPG-strips were equilibrated for 20 min in buffer (6 M urea, 30% v/v glycerol (87% v/v), 2% w/v SDS, 50 mM Tris-Cl, pH 8.8, 100 mg DTT/10 mL, and traces of bromophenol blue). Gels were silver-stained according to Heukeshoven and Dernick⁵² using a silver staining kit (GE Healthcare). Gels were analyzed using the Proteomeweaver software package (Definiens, Germany).

For global protein synthesis detection platelets were cultured in DMEM without L-methionine (#21013-024, Invitrogen,) for 2 hr to deplete any residual L-methionine. During these two hours one part of the sample was also preincubated with puromycin (P-8833, Sigma) as previously described [11]. The medium was then substituted with 250 μM Click-iT AHA (#MP10102, Invitrogen), an amino acid analog of L-methionine containing an azido moiety. Cultured platelets were fixed in suspension with 4% PFA for 20 min. at room temperature and subsequently layered onto Vectabond™ (Vector Laboratories, Burlingame, Calif.) coated coverslips using a cytospin centrifuge (Shandon Cytospin, Thermo Fisher Scientific, Waltham, Mass.). The fixed platelets were washed and permeabilized in 0.25% NP-40 in PBS (15 min. at room temperature). The amino acid analog was detected by using a custom made Alexa Fluor 488 conjugated alkyne

(5 μM). The chemoselective ligation between the azide and alkyne was performed using the Click-iT Protein Analysis Detection Kit (#MP33370, Invitrogen) with a reaction time of 15 min. The binding of fluorescent dye was detected using confocal microscopy. Unlabeled platelets that were fixed at baseline were used as a marker of background fluorescence.

Microscopy. Low-resolution wide-field microscopy used in at least FIG. 1F was performed with a Nikon Eclipse E400 microscope (Nikon Instruments Inc., Melville, N.Y.) equipped with a 40×/0.65 NA objective and a PixeLINK PL-A662 camera (PixeLINK, Ottawa, ON, Canada). Fluorescence microscopy and high resolution confocal reflection microscopy was performed using an Olympus IX81, FV300 (Olympus, Melville, N.Y.) confocal-scanning microscope equipped with a 60×/1.42 NA oil objective for viewing platelets. An Olympus FVS-PSU/IX2-UCB camera and scanning unit and Olympus Fluoview FV 300 image acquisition software version 5.0 were used for recording. The images were further analyzed using Adobe Photoshop CS version 8.0, Metamorph software (Molecular Devices), and ImageJ (NIH). Real-time microscopy was performed using an Olympus IX81 microscope (Olympus, Melville, N.Y.) and images were assessed with Metamorph software. Single frames were further processed using Iprocess (Cell Imaging Core, University of Utah) as well as ImageJ. TEM thin sections (FIG. 2B) were examined with a JOL JEM-1011 electron microscope and digital images were captured with a side-mounted Advantage HR CCD camera (Advanced Microsystems Techniques [AMT], Danvers Mass.). A Hitachi H-7100 transmission electron microscope was used to observe and photograph the thin sectioned cell shown in FIG. 2C.

Platelet Numbers. For the studies displayed in at least FIG. 4D, platelets were resuspended into culture medium and then evenly separated into two samples (baseline or cultured (1×10⁵ per mm³)). In select studies, the cultured platelets were treated with thrombin (0.01 U/ml). The baseline sample was immediately fixed with paraformaldehyde as described above. The remaining sample was incubated in suspension for 6 hours and then fixed in a manner identical to the baseline sample. The volume of each sample was measured and the cells were subsequently counted for exactly two minutes with a flow cytometer. The volume for each sample was then re-measured and subtracted from its corresponding baseline volume to account for variability in sample uptake by the flow cytometer. Cell counts, using time as the variable held constant, were subsequently calculated for each sample. Duplicate samples were performed for each experimental condition to provide a mean cell count for each donor.

Reverse Transcriptase Activity Assay (FIGS. 10A-C and FIGS. 11A-C). The reverse transcriptase (RT) activity assay was performed as previously described²² with minor modifications. Lysed platelets were incubated with nevirapine, 9-cis retinoic acid or vehicle (DMSO) at 37° C. for 120 minutes. MS2 phage RNA was subsequently added to the lysates and reverse transcription of MS2 phage RNA by platelet lysates or a commercial RT (Thermoscript, Invitrogen, Eugene, Oreg.) was determined.

Statistical Analyses. The mean±SEM was determined for each experimental variable displayed in FIGS. 1C, 3A, 4A(II), 4A(III), 4A(IV), 4B, 5B, 8, 9A, 9D, 10C, 11B and 12. ANOVA's were conducted to identify differences that existed among multiple experimental groups. If significant differences were found, a Student-Newman-Keuls post-hoc procedure was used to determine the location of the difference. Paired t-tests were used for comparisons between two groups. For the analysis in FIG. 4B, a paired t-test was used. For the analysis in FIG. 5D, a paired t-test and a two-way ANOVA was used. For all of the analyses, p<0.05 was considered statistically significant.

### Example I

### Platelet Isolation and Culture Conditions for Promoting Extension/Expansion

Whole blood was centrifuged at 150×g for 20 minutes to obtain platelet-rich plasma (PRP). Residual leukocytes were removed from the PRP by CD45+ bead selection as previously described1,6. The supernatant was discarded and the cells were resuspended and pelleted again at 1500 rpm for 20 min. The supernatant was again discarded and the cells were suspended in 50 ml Pipes/saline/glucose buffer (PSG) containing 100 μM prostaglandin E1 (PGE1). Two μl of MACS® CD45 MicroBeads (Miltenyi Biotec, Germany) per ml of original platelet rich plasma volume was added and the solution was incubated for 20 min. at room temperature, mixing periodically. The entire volume of platelets, platelet storage granules, and beads were then placed on an auto-MACS® machine and using the “depletes” program, the beads were separated at 1500 rpm for 20 minutes. The supernatant was discarded and the cells were resuspended in a small volume of warm (37° C.) M199 culture medium.

The negatively selected platelets were resuspended in serum-free M199 culture medium at 37° C. in a humidified 5% CO₂ atmosphere. Washed platelets were cultured in suspension using round-bottom polypropylene tubes (Becton Dickinson, Franklin Lakes, N.J.). Warm M199 media may then be added to obtain the desired concentration of platelet cells, for example a concentration of 1×10⁵ per mm3. Optionally, the cells may then be counted. The platelets are then incubated for the desired time at 37° C. in a humidified atmosphere. Platelets cultured at 1×10⁵ per mm³ in plasma out of the FDA storage bags produce extended cells also (data not shown).

Stored platelets were obtained from the ARUP Blood Transfusion Services at the University of Utah or the Institute of Transfusion Medicine at the University of Greifswald. The apheresed platelets were immediately placed in standardized platelet bags and stored under constant agitation in a climate-controlled chamber (Melco Engineering Corp., Glendale, Calif.) that was maintained between 20-24° C. On day 1 and day 4 (i.e., 24 and 96 hours after apheresis, respectively), samples of the ex vivo aged platelets were removed under sterile conditions, gently washed, and subsequently resuspended as described above.

Apheresed platelets were obtained from healthy blood donors, who had not taken any medication during the previous 10 days, by the apheresis device ComTec (Fresenius GmbH, Bad Homburg, Germany). Platelet concentrates were rested for 2 hours to allow reconstitution of minor platelet activation. Then a separate bag was aligned to the platelet bag tube by sterile docking to obtain a sample of the platelet concentrate. Platelet count and mean platelet volume were determined using an automated particle counter (Sysmex, Sysmex Japan). To reduce counting errors due to high platelet numbers, samples were diluted 1:4 before measuring using PBS-EDTA 2%. Platelet concentrates were stored under agitation as described above for five days and a second sample was obtained for determination of platelet count and mean platelet volume.

### Example II

Platelets isolated from human plasma by apheresis were cultured under mild thrombocytopenic conditions (1×10⁵ per mm³) or under non-thrombocytopenic conditions (1×10⁶ per mm³). After 2 hours under culture conditions, the platelets were pelleted, and the supernatant from the platelets cultured at 1×10⁶ per mm³ was used to resuspend platelets cultured at 1×10⁵ per mm³ and vice versa. The platelets were then cultured for approximately another two hours. The supernatants from the low concentrated platelets were subject to different size exclusions columns (10 kDa and 30 kDa) and the filtrate or the retentate was added to the high-concentrated platelets.

All references, including publications, patents, and patent applications, cited herein are hereby incorporated by reference to the same extent as if each reference were individually and specifically indicated to be incorporated by reference and were set forth in its entirety herein: including;
1. Denis, M. M., et al. Escaping the nuclear confines: signal-dependent pre-mRNA splicing in anucleate platelets. Cell 122, 379-391 (2005).
2. Geddis, A. E. & Kaushansky, K. Immunology. The root of platelet production. Science 317, 1689-1691 (2007).
3. Italiano, J. E., Jr., Lecine, P., Shivdasani, R. A. & Hartwig, J. H. Blood platelets are assembled principally at the ends of proplatelet processes produced by differentiated megakaryocytes. The Journal of cell biology 147, 1299-1312 (1999).
4. Junt, T., et al. Dynamic visualization of thrombopoiesis within bone marrow. Science 317, 1767-1770 (2007).
5. Kaushansky, K. The molecular mechanisms that control thrombopoiesis. The Journal of clinical investigation 115, 3339-3347 (2005).
6. Schwertz, H., et al. Signal-dependent splicing of tissue factor pre-mRNA modulates the thrombogenecity of human platelets. J Exp Med 203, 2433-2440 (2006).
7. Behnke, O. & Forer, A. From megakaryocytes to platelets: platelet morphogenesis takes place in the bloodstream. Eur J Haematol Suppl 61, 3-23 (1998).
8. Hansen, M. & Pedersen, N. T. Circulating megakaryocytes in blood from the antecubital vein in healthy, adult humans. Scand J Haematol 20, 371-376 (1978).
9. Handagama, P. J., Feldman, B. F., Jain, N. C., Farver, T. B. & Kono, C. S. Circulating proplatelets: isolation and quantitation in healthy rats and in rats with induced acute blood loss. Am J Vet Res 48, 962-965 (1987).
10. Tong, M., Seth, P. & Penington, D. G. Proplatelets and stress platelets. Blood 69, 522-528 (1987).
11. Italiano, J., Jr. and Hartwig, J H. Megakaryocyte Development and Platelet Formation. in Platelets (ed. Michelson, A. D.) 21-35 (Elsevier, San Diego, 2002).
12. Clemetson, K. Platelet Receptors. in Platelets (ed. Michelson, A.) 65-84 (Elsevier, Amsterdam, 2002).
13. Coller, B. S. Platelet GPIIb/IIIa antagonists: the first anti-integrin receptor therapeutics. The Journal of clinical investigation 99, 1467-1471 (1997).
14. McEver, R. P. Adhesive interactions of leukocytes, platelets, and the vessel wall during hemostasis and inflammation. Thrombosis and Haemostasis 86, 746-756. (2001).
15. John, G. B., et al. The mitochondrial inner membrane protein mitofilin controls cristae morphology. Mol Biol Cell 16, 1543-1554 (2005).
16. Italiano, J. E., Jr. & Shivdasani, R. A. Megakaryocytes and beyond: the birth of platelets. J Thromb Haemost 1, 1174-1182 (2003).
17. Barr, F. A. & Gruneberg, U. Cytokinesis: placing and making the final cut. Cell 131, 847-860 (2007).
18. Haimovich, B., Lipfert, L., Brugge, J. S. & Shattil, S. J. Tyrosine phosphorylation and cytoskeletal reorganization in platelets are triggered by interaction of integrin receptors with their immobilized ligands. The Journal of biological chemistry 268, 15868-15877 (1993).
19. Kai, Y., et al. Mitochondrial DNA replication in human T lymphocytes is regulated primarily at the H-strand termination site. Biochim Biophys Acta 1446, 126-134 (1999).
20. Guertin, D. A., Trautmann, S. & McCollum, D. Cytokinesis in eukaryotes. Microbiol. Mol Biol Rev 66, 155-178 (2002).
21. Nishitani, H. & Lygerou, Z. Control of DNA replication licensing in a cell cycle. Genes Cells 7, 523-534 (2002).
22. Mangiacasale, R., et al. Exposure of normal and transformed cells to nevirapine, a reverse transcriptase inhibitor, reduces cell growth and promotes differentiation. Oncogene 22, 2750-2761 (2003).
23. Sinibaldi-Vallebona, P., Lavia, P., Garaci, E. & Spadafora, C. A role for endogenous reverse transcriptase in tumorigenesis and as a target in differentiating cancer therapy. Genes Chromosomes Cancer 45, 1-10 (2006).
24. Kubo, S., et al. L1 retrotransposition in nondividing and primary human somatic cells. Proceedings of the National Academy of Sciences of the United States of America 103, 8036-8041 (2006).
25. Spadafora, C. Endogenous reverse transcriptase: a mediator of cell proliferation and differentiation. Cytogenet Genome Res 105, 346-350 (2004).
26. Weyrich, A. S., et al. mTOR-dependent synthesis of Bcl-3 controls the retraction of fibrin clots by activated human platelets. Blood 109, 1975-1983 (2007).
27. Xia, Y., Whitesides, G M. Angew Chem Int Ed 37, 550 (1998).
28. Anna, S. L., Bontoux, N. & Stone, H. A. Applied Physics Letters 82, 364 (2003).
29. Randall, G. C. & Doyle, P. S. Permeation-driven flow in poly(dimethylsiloxane) microfluidic devices. Proceedings of the National Academy of Sciences of the United States of America 102, 10813-10818 (2005).
30. Lowe, K. C., Davey, M. R. & Power, J. B. Perfluorochemicals: their applications and benefits to cell culture. Trends Biotechnol 16, 272-277 (1998).
31. Analysis of platelets from patients with thrombocythemia for reverse transcriptase and virus-like particles. J Natl Cancer Inst. 1975 November; 55(5):1069-74.
32. Lodish H B A, Kaiser C A, Krieger M, Scott M P, Bretscher A, Ploegh H, Matsudaira P. Regulating the Eukaryotic Cell Cycle. In: K A, ed. Molecular Cell Biology (ed 6). New York: W.H. Freeman and Company; 2008:849-902.
33. Mason K D, Carpinelli M R, Fletcher J I, et al. Programmed anuclear cell death delimits platelet life span. Cell. 2007; 128:1173-1186.
34. Trivedi V, Boire A, Tchernychev B, et al. Platelet matrix etalloprotease-1 mediates thrombogenesis by activating PAR1 at a cryptic ligand site. Cell. 2009; 137:332-343.
35. Davi G, Patrono C. Platelet activation and atherothrombosis. N Engl J. Med. 2007; 357:2482-2494.
36. Qian K, Xie F, Gibson A W, Edberg J C, Kimberly R P, Wu J. Functional expression of IgA receptor Fc{alpha}RI on human platelets. J Leukoc Biol. 2008.
37. Shashkin P N, Brown G T, Ghosh A, Marathe G K, McIntyre T M. Lipopolysaccharide is a direct agonist for platelet RNA splicing. J. Immunol. 2008; 181:3495-3502.
38. Brogren H, Karlsson L, Andersson M, Wang L, Erlinge D, Jern S. Platelets synthesize large amounts of active plasminogen activator inhibitor 1. Blood. 2004; 104:3943-3948.
39. Evangelista V, Manarini S, Di Santo A, et al. De novo synthesis of cyclooxygenase-1 counteracts the suppression of platelet thromboxane biosynthesis by aspirin. Circ Res. 2006; 98:593-595.
40. Panes O, Matus V, Saez C G, Quiroga T, Pereira J, Mezzano D. Human platelets synthesize and express functional tissue factor. Blood. 2007; 109:5242-5250.
41. Savini I, Catani M V, Arnone R, et al. Translational control of the ascorbic acid transporter SVCT2 in human platelets. Free Radic Biol Med. 2007; 42:608-616.
42. Weyrich A S, Dixon D A, Pabla R, et al. Signal-dependent translation of a regulatory protein, Bcl-3, in activated human platelets. Proc Natl Acad Sci USA. 1998; 95:5556-5561.
43. Thon J N, Devine D V. Translation of glycoprotein IIIa in stored blood platelets. Transfusion. 2007; 47:2260-2270.
44. Italiano J E, Jr., Lecine P, Shivdasani R A, Hartwig J H. Blood platelets are assembled principally at the ends of proplatelet processes produced by differentiated megakaryocytes. J. Cell Biol. 1999; 147:1299-1312.
45. Yang H, Lang S, Zhai Z, et al. Fibrinogen is required for maintenance of platelet intracellular and cell-surface P-selectin expression. Blood. 2009; 114:425-436.
46. Patel-Hett S, Richardson J L, Schulze H, et al. Visualization of microtubule growth in living platelets reveals a dynamic marginal band with multiple microtubules. Blood. 2008; 111:4605-4616.
47. Italiano J E, Jr., Patel-Hett S, Hartwig J H. Mechanics of proplatelet elaboration. J. Thromb. Haemost. 2007; 5 Suppl 1:18-23.
48. Ebbe S, Stohlman F, Jr., Overcash J, Donovan J, Howard D. Megakaryocyte size in thrombocytopenic and normal rats. Blood. 1968; 32:383-392.
49. Schwertz H, Carter J M, Abdudureheman M, Russ M, Buerke U, et al. (2007) Myocardial ischemia/reperfusion causes VDAC phosphorylation which is reduced by cardioprotection with a p38 MAP kinase inhibitor. Proteomics 7: 4579-4588.
50. Schwertz H, Langin T, Platsch H, Richert J, Bomm S, et al. (2002) Two-dimensional analysis of myocardial protein expression following myocardial ischemia and reperfusion in rabbits. Proteomics 2: 988-995.
51. Popov N, Schmitt M, Schulzeck S, Matthies H (1975) [Reliable micromethod for determination of the protein content in tissue homogenates]. Acta Biol Med Ger 34: 1441-1446.
52. Heukeshoven J, Dernick R (1988) Improved silver staining procedure for fast staining in PhastSystem Development Unit. I. staining of sodium dodecyl sulfate gels. Electrophoresis 9: 28-32.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of increasing the number of platelets in a preparation of anucleate platelets, the method comprising:
diluting a preparation of platelets with media; and
culturing the diluted platelets to produce additional platelets.

2. The method according to claim 1, comprising culturing the platelets under conditions that mimic thrombocytopenic conditions.

3. The method according to claim 2, wherein the platelets are cultured at concentration of at or less than about 1×10⁸ cells per mm³.

4. The method according to claim 3, wherein the platelets at cultured at 37° C. for a period of from about 24 hours to about 96 hours.

5. The method according to claim 1, comprising resuspending the cultured platelets in fresh human plasma.

6. The platelet preparation of claim 1, wherein the platelets are cultured in M199 media.

7. The method according to claim 1, further comprising adding a reverse transcriptase inhibitor to the diluted platelets.

8. The method according to claim 7, wherein the reverse transcriptase inhibitor is a non-nucleoside inhibitor.

9. The method according to claim 8, wherein the reverse transcriptase inhibitor is selected from the group consisting of Nevirapine, Delavirdine, Evafirenz, Etravirine, and combinations thereof.

10. The method according to claim 7, wherein the reverse transcriptase inhibitor is a nucleoside inhibitor.

11. The method according to claim 10, wherein the reverse transcriptase inhibitor is selected from the group consisting of AZT, ddI, ddC, d4T, 3TC, ABC, FTC, and combinations thereof.

12. The method according to claim 1, further comprising adding a modulator of the retinoic acid receptor X activity to the diluted platelets.

13. The method according to claim 12, wherein the modulator of the retinoic acid receptor X activity is 9-cis retinoic acid.

14. A method of treating a thrombocytopenia condition in a subject, the method comprising:
administering to a subject an effective amount of an agent which induces proliferation of platelets from anucleate platelets; and
inducing production of daughter platelets from platelets present in the subject.

15. The method according to claim 14 wherein said agent that induces proliferation of platelets is a reverse transcriptase inhibitor.

16. The method according to claim 14, further comprising adding a modulator of the retinoic acid receptor X activity to the diluted platelets.

17. The method according to claim 14 wherein said agent is a soluble protein derived from the supernatant of cultured anucleate platelets.

18. A platelet preparation comprising platelets cultured under thrombocytopenic conditions from anucleate platelets in a preparation.

19. The platelet preparation according to claim 18 wherein said preparation is cultured ex vivo.

20. The platelet preparation according to claim 18 wherein the platelets are cultured from freshly-isolated anucleate platelets.

21. The platelet preparation according to claim 18 wherein the platelets are cultured for greater than twenty-four hours.
